(19) United States
(12) Patent Application Publication
(10) Pub. No.: US 2004/0081988 A1
(43) Pub. Date: Apr. 29, 2004

(54) Splice variant isoforms of human calcium channel CACNA1B

(76) Inventors: Jason M. Johnson, Seattle, WA (US); Christopher D. Armour, Kirkland, WA (US); John C. Castle, Seattle, WA (US)

Correspondence Address: R. Douglas Bradley, Merck & Co., Inc., Patent Department RY60-30, P.O. Box 2000, Rahway, NJ, 07065-0907, US

(21) Appl. No.: 10/627,370

(22) Filed: Jul. 25, 2003

Related U.S. Application Data
(60) Provisional application No. 60/400,003, filed on Jul. 30, 2002.
(60) Provisional application No. 60/421,220, filed on Oct. 25, 2002.
(60) Provisional application No. 60/452,258, filed on Mar. 4, 2003.

(51) Int. Cl. C12Q 1/68; C07H 21/04; C07K 14/705

(52) U.S. Cl. 435/6; 435/69.1; 435/320.1; 435/325; 530/350; 536/23.2

(57) ABSTRACT

The present invention features nucleic acids and polypeptides encoding two novel splice variant isoforms of calcium channel α_{1B} subunit (CACNA1B). The polynucleotide sequence of CACNA1Bsv1 is provided by SEQ ID NO 1 and the polynucleotide sequence of CACNA1Bsv2 is provided by SEQ ID NO 3. The amino acid sequence for CACNA1Bsv1 is provided by SEQ ID NO 2 and the amino acid sequence for CACNA1Bsv1 is provided by SEQ ID NO 4. The present invention also provides methods for using CACNAlBsv1 and CACNA1Bsv2 polynucleotides and the respective proteins to screen for compounds that bind to CACNA1Bsv1 and CACNA1Bsv2.

### BACKGROUND OF THE INVENTION

In the nervous system, voltage-dependent channels, also known as voltage-gated channels, regulate the rapid entry of ions from the extracellular environment into the cells and control a variety of cell physiological processes that are related to the neurotransmitter release and neural firing patterns. In addition, calcium channels play an important role in a number of vital processes, including neurotransmitter release, muscle contraction, pacemaker activity, and the secretion of hormones and other substances (U.S. Pat. No. 6,096,514).

The voltage-gated channel proteins are typically multi-subunit proteins containing α₁, α₂, β and γ subunits. The channels are members of the superfamily of ion channel proteins that include voltage-gated Na⁺ channels, K⁺ channels and Ca²⁺ channels. Calcium voltage-gated channels permit the entry of calcium when there is an electrical potential differential between the outside and inside of a cell. There are several classes of calcium channels: N-type, P-Q type, L-type, R-type and T-type channels. However, the distinctions between the different classes of calcium channels may not be clearcut because, for example, varying combinations of P type and Q type channels can arise as the result of alternative splicing of the RNA transcript encoding the α_{1A} subunit RNA (see below).

Alternative RNA splicing is associated with more than a dozen human diseases (Grabowski and Black 2001 Progress in Neurobiology, 65, 289-308, Pergamon Press, N.Y.). Schizophrenia (Huntsman et. al. 1998 Proc. Natl. Acad. Sci. U.S.A. 95, 15066-15071), Myotonic Dystrophy (Phillips et al., 1998, Science, 280, 737-741), and Human Melanoma (skin tumors) (Ge et al., 1999, Proc. Natl. Acad. Sci. U.S.A. 96, 9689-9694) are examples of disease pathologies associated with irregularities in alternative splicing. Defects in the one calcium ion channel gene (CACNA1A) have been linked to neuronal disorders such as familial hemiplegic migraines and episodic ataxia (Ophoff, R., et al., 1996 Cell, 87, 543-552).

The α₁ polypeptide is the largest of the calcium channel subunits having a molecular mass of 190 kDa to 250 kDa. The α₁ subunit forms the essential structural framework of a Ca²⁺ channel comprising the conduction pore, the voltage-sensor, gating apparatus and also contains amino acid domains that interact with secondary messengers and toxins. In some cases, the α₁ polypeptide alone is enough to form a functional calcium channel. In addition, different types of Ca²⁺ channels result from assembly of the α₂, β and γ subunits with the many different isoforms of the α₁ subunit (Ertel et al., 2000, Cell 25, 533-535). For example, molecular cloning has revealed that there are at least ten different α₁ genes (designated CACNA). Experimental analysis of the mRNAs transcribed from these genes has established many examples of alternate splicing, further increasing the number of potentially different calcium channels isoforms. However, the large size of the α₁ transcript (6 kb or more) has made transcript analysis difficult (Beam, K., 1999 Nature Neuroscience 2, 393-394), thereby limiting identification of all splice variants.

N-type Ca 2+ channels comprise an α_{1B}-subunit (CACNA1B, alternatively referred to as Caᵥ2.2). The N-type Ca²⁺ channel is characterized by its sensitivity to ω-conotoxin (ω-CgTx) and insensitivity to dihydropyridine (1,4 DHP). Knockout mice lacking the gene encoding the α_{1B} subunit exhibited an absence of N-type electrical currents and a complete elimination of sensitivity to ω-conotoxin GVIA, but surprisingly, they exhibited otherwise normal mouse behavior. However, the mutant mice did exhibit substantially elevated blood pressure and heart rates. These results provide direct evidence that N-type voltage-dependent Ca²⁺ channels are essential for the normal function of the sympathetic nervous system (Mori et al., 2002, Trends Cardiovasc. Med. 12, 270-275; Ino et al., 2001, Proc. Natl. Acad. Sci. U.S.A. 98, 5323-5328).

The earliest evidence for the heterogeneity of N-type of Ca²⁺ channel was obtained upon identification of α_{1B} splice variants (Williams et al. 1992 Science 257, 389-395). Williams et al. identified two variants, α_{1b-1} and α_{1b-2}, in human neuroblastoma cells. These variant N-type Ca²⁺ channels were found to be irreversibly blocked by ω-CgTx toxin, but were insensitive to dihydropyridines. In addition, cDNA clones encoding other human N-type Ca²⁺ channel variants were isolated and found to be lacking large parts of domains II-III linker region of the α_{1B} subunit termed Caᵥ2.2Δ1 and Δ2 (see FIG. 1), which has been shown to interact with presynaptic protein (Kaneko, et al., 2002, J. Neuroscience, 22, 82-92). Transfection of clones encoding these splice variants into human embryonic kidney (HEK) tsA-201 cells revealed that the splice variant isoforms exhibited different levels of currents as compared to cells not expressing the splice variant isoform alpha subunits (Kaneko et al., 2002, J. Neuroscience, 22, 82-92).

Zhong et al. isolated two isoforms of the α1_{B} subunit from rat brain. These isoforms were termed Caᵥ2.2a and Caᵥ2.2b. Normally, the Caᵥ2.2b calcium channel is in its inactive or resting (closed) state and is easily activable (i.e., willing state). However, in the presence of G-protein β_{γ} subunits (a GTP-binding protein signal transducer), this calcium channel isoform is converted into a so called “reluctant” state, wherein its activation is slowed. In contrast, the Caᵥ2.2a variant was found to exhibit contrasting properties, i.e., the unactivated channel was slow to be activated and was not influenced by the presence or absence G-protein. Thus, the reluctant state of Ca²⁺ channels comprising the Caᵥ2.2a isoform is an intrinsic protein property rather than a property resulting from G-protein interaction. Interestingly, mutation of glycine 177 to glutamic acid residue in the transmembrane segment IS3 (see FIG. 1) of Caᵥ2.2b isoform converts it to a tonically reluctant state. Zhong et al. have proposed that the negatively charged glutamate residue at position 177 of the protein interacts with a positively charged side chain in the IS4 voltage sensor domain, to produce the reluctant state of the Caᵥ2.2a isoform (Zhong et al., 2001 Proc. Natl. Acad. Sci. U.S.A. 98, 4705-4709).

Alternative RNA splice variants of CACNA1B mRNA affecting the extracellular loop regions of the α_{1B} protein subunit domain 111S3-S4 and the domain IVS3-S4 (see FIG. 1) were shown to exhibit differential electrophysiological properties when expressed in brain as compared with peripheral ganglia. The brain-expressed isoform was 2 to 4 fold more rapidly gated than the ganglia-expressed isoform (Lin et al., 1997 Neuron 18, 153-166).

The ET (glutamic acid-threonine) region or EF (glutamic acid-phenylalanine) region in the IVS6 domain (see FIG. 1) is located near the voltage-sensing center of the ion channel, which is thought to be the reason for the extreme sensitivity of this region to amino acid substitutions. Replacement of ET amino acids with NP (asparagine-proline) did not change the kinetics of activation, demonstrating that the side-chains of the ET are not required for slow activation of the channel (Lin et al., 1999 J. Neuroscience, 19, 5322-5331). Two functionally distinct splice variant isoforms of the α_{1B} subunit, referred to as rnα_{1B-b} and rnα_{1B-d}, have been identified. The rnα_{1B-b} splice isoform protein has four amino acids (serine-phenylalanine-methionine-glycine) in the IIS3-S4 region (see FIG. 1) that are different from the normal CACNA1B protein. Isoform rnα_{1B-d} was found to have two amino acids (ET) in IVS3-S4 region that were altered. Both of these splice variant isoforms exhibited slow activation and inactivation properties.

As the foregoing background information indicates, Ca²⁺ channel activity plays an important role in the transmission of nerve impulses by regulating Ca²⁺ ion flow across cell membranes. In addition, Ca²⁺ channel proteins have been causally implicated in several human diseases, such as, familial hemiplegic migraine, episodic ataxia type-2, Lambert-Eton myasthenic syndrome, progressive ataxia, juvenile mytonic epilepsy, malignant hypothermia, hypokalemic periodic paralysis and X-linked congenital stationary night blindness. To study the structure and function of isoform variants of calcium channels and their potential role in disease causation, it is informative to isolate, purify and characterize as many Ca²⁺ channel polypeptide isoforms, and corresponding encoding polynucleotides, as possible. In particular, individual protein subunits of calcium channels, including splice variant isoforms, represent useful reagents in the screening of compounds to identify new therapeutic agents. Furthermore, purified variant channel proteins and polynucleotides are also useful in methods to classify calcium channel ligand compounds based upon isoform specificity. In particular, methods and reagents are need in the art to select therapeutic compounds that are highly specific for the target of calcium channel proteins yet do not bind to or alter the function of off-target polypeptides. In addition, large amounts of purified calcium channel proteins are also required for functional characterization of calcium channel isoforms. Native cells and tissues are unfit for this purpose because native biological materials often have a mixture of calcium channels, rendering the study of individual calcium channel isoforms using single channel recording methods virtually impossible. Thus, there is a need in the art for a comprehensive selection of polynucleotides that encode as many different isoforms of human α₁ protein subunits of the Ca²⁺ channel as can be identified.

### SUMMARY OF THE INVENTION

Genomic tiling microarrays and RT-PCR have been used to identify and confirm the presence of two human splice variants of CACNA1B mRNA. More specifically, the present invention features polynucleotides encoding two different protein isoforms of CACNA1B. The polynucleotide sequence encoding CACNA1Bsv1 is provided by SEQ ID NO 1. The amino acid sequence for CACNA1Bsv1 is provided by SEQ ID NO 2. The polynucleotide sequence encoding CACNA1Bsv2 is provided by SEQ ID NO 3. The amino acid sequence for CACNA1Bsv2 is provided by SEQ ID NO 4.

Thus, a first aspect of the present invention describes a purified CACNA1Bsv1 encoding nucleic acid and a purified CACNA1Bsv2 encoding nucleic acid. The CACNA1Bsv1 encoding nucleic acid comprises SEQ ID NO 1 or the complement thereof. The CACNA1Bsv2 encoding nucleic acid comprises SEQ ID NO 3 or the complement thereof. Reference to the presence of one region does not indicate that another region is not present. For example, in different embodiments the nucleic acid can comprise or consist of a nucleic acid encoding for SEQ ID NO 1, or alternatively, can comprise or consist of the nucleic acid sequence of SEQ ID NO 3.

Another aspect of the present invention describes a purified polypeptide selected from the group consisting of CACNA1Bsv1 and CACNA1Bsv2. Thus, in one embodiment, the CACNA1B polypeptide can comprise or consist of the amino acid sequence of SEQ ID NO 2. In another embodiment, the CACNA1B polypeptide can comprise or consist of the amino acid sequence of SEQ ID NO 4.

Another aspect of the present invention describes two expression vectors. In one embodiment of the invention, the inventive expression vector comprises a nucleotide sequence encoding a polypeptide comprising or consisting of SEQ ID NO 2, wherein the nucleotide sequence is transcriptionally coupled to an exogenous promoter. In another embodiment, the inventive expression vector comprises a nucleotide sequence encoding a polypeptide comprising or consisting of SEQ ID NO 4, wherein the nucleotide sequence is transcriptionally coupled to an exogenous promoter.

Another aspect of the present invention describes recombinant cells comprising expression vectors comprising or consisting of the above-described sequences and the promoters are recognized by RNA polymerase present in the cell. Another aspect of the present invention, describes recombinant cells made by a process comprising the step of introducing into the cell an expression vector comprising a nucleotide sequence comprising or consisting of SEQ ID NO 1, SEQ ID NO 3, or a nucleotide sequence encoding a polypeptide comprising or consisting of an amino acid sequence of SEQ ID NO 2 or SEQ ID NO 4, wherein the nucleotide sequence is transcriptionally coupled to an exogenous promoter. The inventive expression vectors can be used to insert recombinant nucleic acids into the host genome or can exist as autonomous pieces of nucleic acid.

Another aspect of the present invention describes a method of producing CACNA1Bsv1 or CACNA1Bsv2 polypeptides comprising SEQ ID NO 2 or SEQ ID NO 4, respectively. The method involves the step of growing recombinant cells containing an inventive expression vector under conditions wherein the encoded polypeptide is expressed from the expression vector.

Another aspect of the present invention features a purified antibody preparation comprising an antibody that binds selectively to CACNA1Bsv1 as compared to CACNA1B polypeptide that is not CACNA1Bsv1. In another embodiment, a purified antibody preparation is provided comprising antibody that binds selectively to CACNA1Bsv2 as compared to CACNA1B polypeptide that is not CACNA1Bsv1.

Another aspect of the present invention provides a method of screening for compounds that bind to either CACNA1Bsv1, CACNA1Bsv2, or fragments thereof. In one embodiment, the method comprises the steps of: (a) expressing a polypeptide comprising SEQ ID NO 2, from a recombinant nucleic acid; (b) providing to said polypeptide a test preparation comprising one or more compounds; and (c) measuring the ability of said test preparation to bind to said polypeptide. In another embodiment of invention, the above method is performed using a polypeptide comprising SEQ ID NO 2.

In another embodiment of the method, a compound is identified that binds selectively to CACNA1Bsv1 polypeptide as compared to CACNA1B polypeptide that is not CACNA1Bsv1. This method comprises the steps of: providing CACNA1Bsv1 polypeptides comprising SEQ ID NO 2; providing CACNA1B polypeptide that is not CACNA1Bsv1, contacting said CACNA1Bsv1 polypeptide and said CACNA1B polypeptide that is not CACNA1Bsv1 with a test preparation comprising one or more test compounds; and determining the binding of said test preparation to said CACNA1Bsv1 polypeptide and said CACNA1B polypeptide that is not CACNA1Bsv1, wherein a test preparation that binds said CACNA1Bsv1 polypeptide but does not bind said CACNA1B polypeptide that is not CACNA1Bsv1 is a compounds that selectively bind said CACNA1Bsv1. Alternatively, the same method can be performed using CACNA1Bsv2 polypeptide comprising or consisting of SEQ ID NO 4.

Another embodiment of the method, a compound is identified that binds selectively to CACNA1Bsv2 as compared to CACNA1B. This method comprises the steps of: providing CACNA1Bsv2 polypeptides comprising SEQ ID NO 4; providing a CACNA1B polypeptide that is not CACNA1Bsv2, contacting said CACNA1Bsv2 polypeptides and said CACNA1B polypeptide that is not CACNA1Bsv2 with a test preparation comprising one or more test compounds; and determining the binding of said test preparation to said CACNA1Bsv2 polypeptide and said CACNA1B polypeptide that is not CACNA1Bsv2, wherein a compound which binds said CACNA1Bsv2 polypeptide but does not bind the said CACNA1B polypeptide that is not CACNA1Bsv2 is a compound which selectively bind said CACNA1Bsv2 or CACNA1Bsv2 polypeptides.

In another embodiment of the invention, a method is provided for screening for a compound able to bind to or interact with a CACNA1Bsv1 protein or a fragment thereof comprising the steps of: expressing a CACNA1Bsv1 polypeptide comprising SEQ ID NO 2 or a fragment thereof from a recombinant nucleic acids; providing to said polypeptide a labeled CACNA1B ligand that binds to said polypeptide and a test preparation comprising one or more compounds; and measuring the effect of said test preparation on binding of said labeled CACNA1B ligand to said polypeptide, wherein a test preparation that alters the binding of said labeled CACNA1B ligand to said polypeptide contains a compound that binds to or interacts with said polypeptide. In an alternative embodiment, the method is performed using CACNA1Bsv2 polypeptide comprising or consisting of SEQ ID NO 4 or a fragment thereof.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE FIGURES

FIG. 1 illustrates a structural model of the CACNA1B protein embedded in a plasma membrane of a cell. Transmembrane domains I through IV are each composed of six helices regions (S1 through S6, from left to right) and are illustrated as vertical cylinders. A portion of CACNA1B transmembrane helix IVS6 and an intracellular region designated as the “EF region” are missing from the CACNA1Bsv1 isoform protein. The missing region is shown in gray.

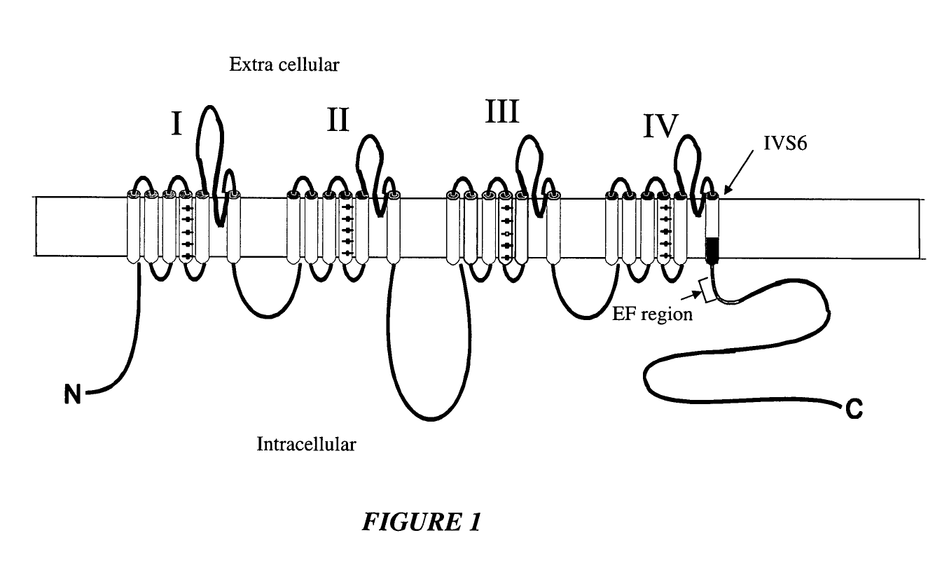

FIG. 2A illustrates the exon structure of CACNA1B mRNA corresponding to the known long form of CACNA1B mRNA (labeled NM_{—000718}). FIG. 2B illustrates the inventive short form splice variant of CACNA1Bsv1 mRNA. The small horizontal arrows above exons 19 and 25 in FIGS. 2A and 2B show the positions of the oligonucleotide primers used to perform RT-PCR assays to confirm the exon structure of CACNA1B mRNA in 40 tissue samples. The nucleotide sequences shown in boxes below the exon structure diagrams of the CACNA1B mRNAs depict the nucleotides sequences of the exon junctions resulting from the splicing of exon 20 to exon 21 and exon 22 to exon 23 in the case of the CACNA1B mRNA (FIG. 2A). In the case of CACNA1Bsv1, exons 21 and 22 are missing (FIG. 2B). In FIGS. 2A and 2B, the nucleotides shown in italics represent the 20 nucleotides located at the 3′ end of exon 20 and the nucleotides shown in underline represent the 20 nucleotides located at the 5′ end of exon 23. In FIG. 2A, the boldface nucleotides associated with the exons 20 to 21 junction represent the 20 nucleotides located at the 5′ end of exon 23, while the boldface nucleotides associated with the exon 22 to exon 23 splice junction represent the 20 nucleotides located at the 3′ end of exon 20.

FIG. 3A illustrates the exon structure of CACNA1B mRNA corresponding to the known long form of CACNA1B mRNA (labeled NM_{—}000718). FIG. 3A illustrates the inventive short form splice variants of CACNA1Bsv2 mRNA (labeled CACNA1Bsv2). The small horizontal arrows above exons 19 and 25 in the FIGS. 3A and 3B show the positions of the oligonucleotide primers used to perform RT-PCR assays to confirm the exon structure of CACNA1B mRNA in 40 human and monkey tissue samples. The nucleotide sequences shown in boxes below the exon structure diagrams of the CACNA mRNAs depict the nucleotides sequences of the exon junctions resulting from the splicing of exon 21 to exon 22 and exon 22 to exon 23 in the case of the CACNA1B mRNA (FIG. 3A). In the case of CACNA1Bsv2 mRNA exon 22 is missing (FIG. 3B). In FIGS. 3A and 3B, the nucleotides shown in italics represent the 20 nucleotides located at the 3′ end of exon 20 and the nucleotides shown in underline represent the 20 nucleotides located at the 5′ end of exon 23. In FIG. 3A, the boldface nucleotides associated with the exon 21 to exon 22 junction represent the 20 nucleotides located at the 5′ end of exon 22, while the boldface nucleotides associated with the exon 22 to exon 23 splice junction represent the 20 nucleotides located at the 3′ end of exon 22.

Definitions

Unless defined otherwise, all technical and scientific terms used herein have the meaning commonly understood by one of ordinary skill in the art to which this invention belongs.

As used herein, “CACNA1B” refers to Ca²⁺ channel Caᵥ2.2 protein subunit 1B (NP_{—000709}). In some contexts of usage the term is meant to include CACNA1B isoforms having amino acid sequences that are not identical to NP_{—000709}.

As used herein, “CACNA1Bsv1” and “CACNA1Bsv2” refer to first and second protein isoforms, respectively, of CACNA1B protein having an amino acid sequence set forth in SEQ ID NO 2 and SEQ ID NO 4, respectively.

As used herein, “CACNA1B” refers to polynucleotides encoding CACNA1B and isoforms thereof.

As used herein, “CACNA1Bsv1” refers to polynucleotides encoding CACNA1Bsv1 having an amino acid sequence set forth in SEQ ID NO 1. “CACNA1Bsv2” refers to polynucleotides encoding CACNA1Bsv1 having an amino acid sequence set forth in SEQ ID NO 3.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction has a dissociation constant of less than 10⁻⁷ M, with specific binding reactions of greater specificity typically having affinity or avidity of at least 10⁻⁸ M to at least about 10⁻⁹ M.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

The present invention relates to the nucleic acid sequences encoding human CACNA1Bsv1 and CACNA1Bsv2 polypeptides, which are splice variant isoforms of CACNA1B, and to amino acid sequences encoding these proteins. SEQ ID NO 1 and SEQ ID NO 3 are polynucleotide sequences representing the full open reading frames that encode CACNA1Bsv1 protein and CACNA1Bsv2 protein, respectively. SEQ ID NO 2 shows the polypeptide sequence of CACNA1Bsv1 and SEQ ID NO 4 shows the polypeptide sequence of CACNA1Bsv2.

CACNA1Bsv1 and CACNA1Bsv2 polynucleotide sequences encoding CACNA1Bsv1 and CACNA1Bsv2 proteins, respectively, as exemplified and enabled herein include a number of specific, substantial and credible utilities. For example, CACNA1Bsv1 and CACNA1Bsv2 encoding nucleic acids were identified in mRNA samples obtained from human sources (see Example 1-3). Such nucleic acids can be used as hybridization probes to distinguish between cells that produce CACNA1Bsv1 and CACNA1Bsv2 transcripts from human or non-human cells (including bacteria) that do not produce such transcripts. Similarly, antibodies specific for CACNA1Bsv1 or CACNA1Bsv2 proteins can be used to distinguish between cells that express CACNA1Bsv1 or CACNA1Bsv2 proteins from human or non-human cells (including bacteria) that do not express CACNA1Bsv1 or CACNA1Bsv2 proteins.

CACNA1B and isoforms thereof, are important drug target for the function of the sympathetic nervous system or for the management of neurodegenerative and cardiovascular disorders. Given the importance of CACNA1B activity to the therapeutic management of pain levels associated with these diseases, it is important to identify CACNA1B isoforms and identify CACNA1B-ligand compounds that are isoform specific as well as compounds that are effective ligands for all CACNA1B isoforms. In particular, it may be important to identify compounds that are effective inhibitors of CACNA1B activities, but do not bind or interact with all CACNA1B isoforms, such as, for example, CACNA1Bsv1 and CACNA1Bsv2. Compounds that bind or interact with all CACNA1B isoforms may require higher drug doses to saturate all CACNA1B-isoform binding sites and thereby achieve a therapeutic benefit. Higher drug doses are well known to increase the likelihood of secondary non-therapeutic side effects. For the foregoing reasons, CACNA1Bsv1 and CACNA1Bsv2 proteins represent important targets for compounds that bind or interact with CACNA1Bsv1 and CACNA1Bsv2 proteins and have utility in the identification of new CACNA1B-interacting compounds having greater specificity and efficacy.

In some embodiments, CACNA1Bsv1 or CACNA1Bsv2 activities are modulated by ligand compounds to achieve proper functioning of the sympathetic nervous system or to prevent, or reduce the risk of occurrence or reoccurrence of a cardiovascular or neurodegenerative disorder. Compounds that affect the nervous system are particularly important for the treatment of pain in the context of these diseases or cancer (For a review, Catterall and Mackie, In, Goodman & Gilman's The Pharmacological Basis of Therapeutics, 9th Ed., McGraw-Hill, New York, 1996, Ch. 15, pp. 367-384).

CACNA1Bsv1 or CACNA1Bsv2 activities can also be affected by modulating the cellular abundance of transcripts encoding CACNA1Bsv1 or CACNA1Bsv2. Compounds modulating the abundance of transcripts encoding CACNA1Bsv1 or CACNA1Bsv2 include cloned polynucleotides comprising CACNA1Bsv1 or CACNA1Bsv2 coding sequences that can be used to express CACNA1Bsv1 or CACNA1Bsv2 in vivo, antisense nucleic acids targeted to CACNA1Bsv1 or CACNA1Bsv2 transcripts and inhibitory ribonucleic acids, such as ribozymes and RNAi, targeted to CACNA1Bsv1 or CACNA1Bsv2 transcripts.

In some embodiments, CACNA1Bsv1 or CACNA1Bsv2 activities are modulated to achieve a therapeutic effect upon diseases in which neurotransmission is in need of adjustment in subjects. For example, neurodegenerative and cardiovascular disorders and abnormalities of the sympathetic nervous system, may be treated by modulating CACNA1Bsv1 or CACNA1Vsv2 activities.

CACNA1Bsv1 AND CACNA1Bsv2 Nucleic Acids

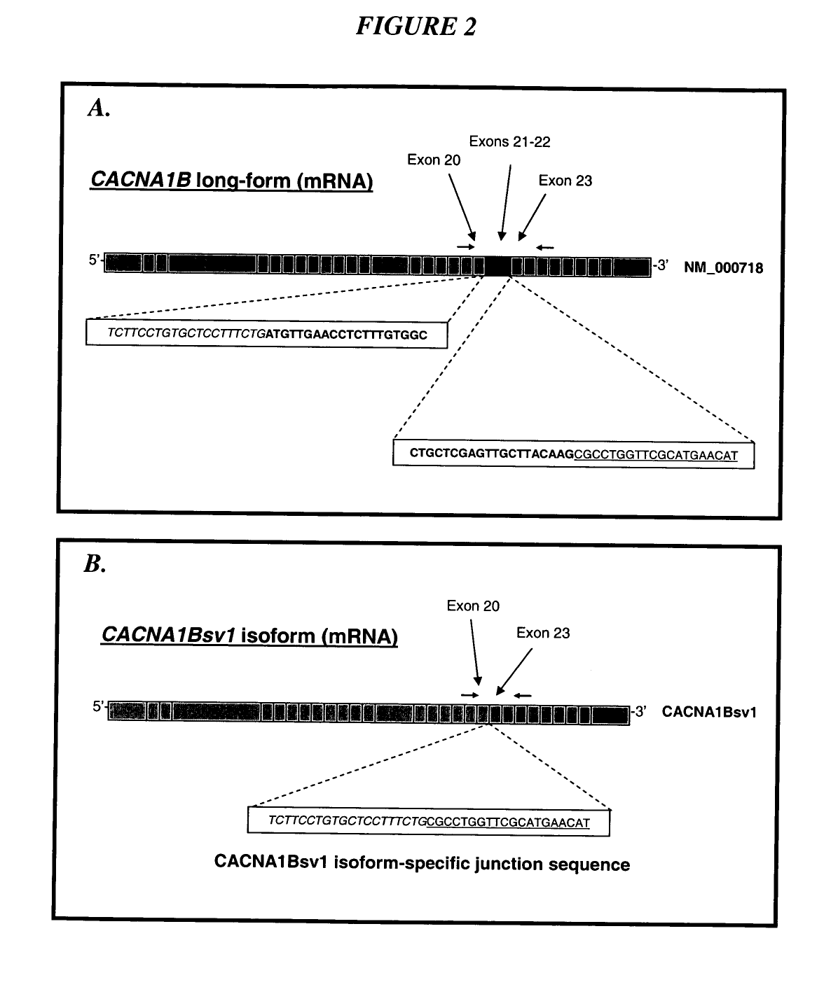

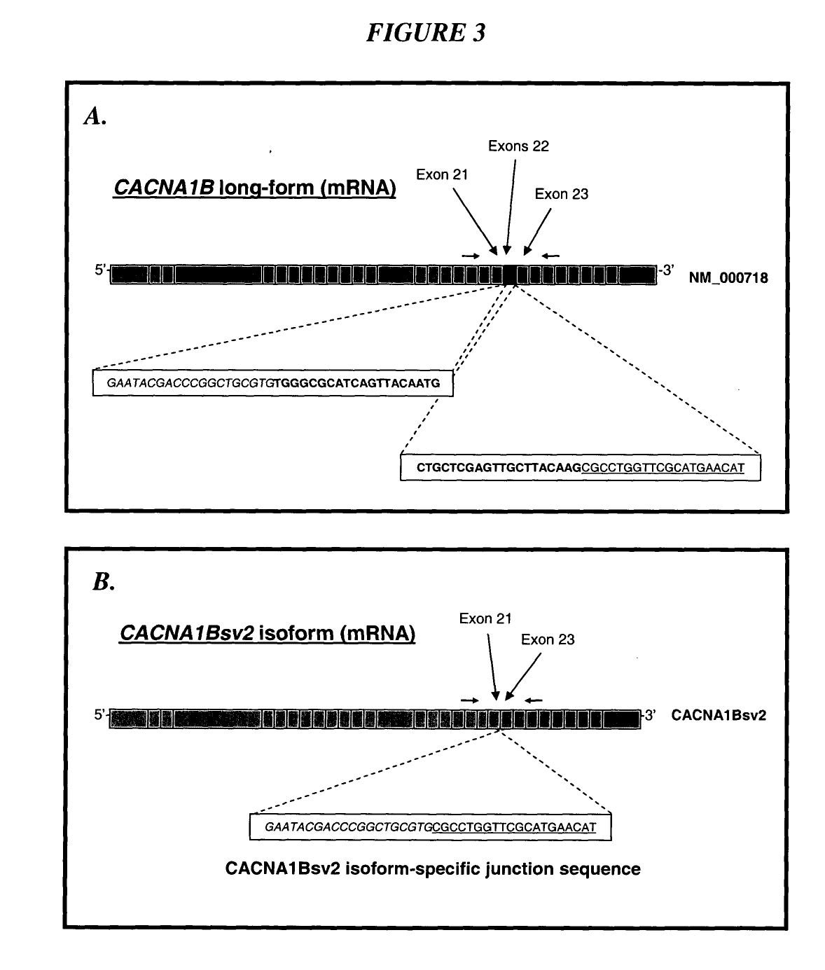

CACNA1Bsv1 nucleic acids contain regions that encode for polypeptides comprising or consisting of SEQ ID NO 2. CACNA1Bsv2 nucleic acids contain regions that encode for polypeptides comprising or consisting of SEQ ID NO 4. The CACNA1Bsv1 and CACNA1Bsv2 nucleic acids have a variety of uses, such as being used as a hybridization probe or PCR primer to identify the presence of CACNA1Bsv1 or CACNA1Bsv2 nucleic acids; being used as hybridization probes or PCR primers to identify nucleic acid encoding for proteins related to CACNA1Bsv1 or CACNA1Bsv2; and/or being used for recombinant expression of CACNA1Bsv1 or CACNA1Bsv2 polypeptides. In particular, CACNA1Bsv1 polynucleotides do not have the polynucleotide regions that comprise exons 21 and 22 of the CACNA1B gene (see FIGS. 2A and 2B). Similarly, CACNA1Bsv2 polynucleotides do not have the polynucleotide regions that comprises exon 22 of the CACNA1B gene (see FIGS. 3A and 3B).

Regions in CACNA1Bsv1 or CACNA1Bsv2 nucleic acids that do not encode for CACNA1Bsv1 or CACNA1Bsv2 amino acids, respectively, are not shown in SEQ ID NO 1 and SEQ ID NO 3, respectively, and if present, are preferably chosen to achieve a particular purpose. Examples of additional regions that can be used to achieve a particular purpose include capture regions that can be used as part of a sandwich assay, reporter regions that can be probed to indicate the presence of the nucleic acid, expression vector regions, and regions encoding for other polypeptides.

The guidance provided in this application can be used to obtain nucleic acid sequences encoding for CACNA1Bsv1 or CACNA1Bsv2-related proteins from different sources. Obtaining nucleic acids encoding for CACNA1Bsv1 or CACNA1Bsv2-related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

CACNA1Bsv1 or CACNA1Bsv2 probes and primers can be used to screen nucleic acid libraries containing, for example, cDNA. Such libraries are commercially available, and can be produced using techniques such as those described in Ausubel, Current Protocols in Molecular Biology, John Wiley, 19871998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asn=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Nucleic acids having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be purchased from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

CACNA1Bsv1 or CACNA1Bsv2 Probes

Probes for CACNA1Bsv1 or CACNA1Bsv2 contain a region that can specifically hybridize to CACNA1Bsv1 or CACNA1Bsv2 target nucleic acids respectively, under appropriate hybridization conditions and can distinguish CACNA1Bsv1 or CACNA1Bsv2 nucleic acids from non-target nucleic acids, in particular CACNA1B polynucleotides representing exons 21 and 22. Probes for CACNA1Bsv1 or CACNA1Bsv2 can also contain nucleic acid regions that are not complementary to CACNA1Bsv1 or CACNA1Bsv2 nucleic acids, respectively.

In embodiments where, for example, CACNA1Bsv1 or CACNA1Bsv2 polynucleotide probes are used in hybridization assays to specifically detect the presence of CACNA1Bsv1 or CACNA1Bsv2 polynucleotides in samples, the CACNA1Bsv1 or CACNA1Bsv2 polynucleotides comprise at least 20 nucleotides of a sequence that corresponds to the respective novel exon junction polynucleotide regions. In particular, for detection of CACNA1Bsv1 polynucleotides the probes comprise at least 20 nucleotides of the CACNA1Bsv1 sequence that corresponds to an exon junction polynucleotide region created by the alternative splicing of exon 20 to exon 23 of the primary transcript of the CACNA1B gene (see FIGS. 2A and 2B). For example, the polynucleotide sequence: 5′-TCTTCCTGTGCTCCTTTCTCGCCTGG TTCGCATGAACAT-3′ [SEQ ID NO 5] represents one embodiment of such an inventive CACNA1Bsv1 polynucleotide probe wherein a first 20 nucleotides region is complementary and hybridizable to the 3′ end of exon 20 of the CACNA1B gene and a second 20 nucleotide region is complementary and hybridizable to the 5′ end of exon 23 of the CACNA1B gene (see FIG. 2B).

For embodiments involving detection of CACNA1Bsv2 encoding splice variant polynucleotides, the CACNA1Bsv2 polynucleotide probes comprise at least 20 nucleotides of the CACNA1Bsv2 sequence that corresponds to a junction polynucleotide region created by the alternative splicing of exon 21 to exon 23 of the primary transcript of the CACNA1B gene (see FIGS. 3A and 3B). For example, the polynucleotide sequence: 5′-GAATACGACCCGGCTGCGTGCGCCTGGTTCGC ATGAACAT-3′ [SEQ ID NO 6] represents one embodiment of such an inventive CACNA1Bsv2 polynucleotide probe wherein a first 20 nucleotides region is complementary and hybridizable to the 3′ end of exon 21 of the CACNA1B gene and a second 20 nucleotide region is complementary and hybridizable to the 5′ end of exon 23 of the CACNA1B gene (see FIG. 3B).

In some embodiments of the CACNA1Bsv1 or CACNA1Bsv2 probes, the at least 20 nucleotides of CACNA1Bsv1 or CACNA1Bsv2 splice junction nucleotides comprises a first continuous region of 5 to 20 nucleotides that is complementary and hybridizable to the 3′ end of exon 20 or exon 21, respectively, and a second continuous region of 5 to 20 nucleotides that is complementary and hybridizable to the 5′ end exon 23.

In other embodiments, the CACNA1Bsv1 or CACNA1Bsv2 polynucleotides comprise at least 40, 60, 80 or 100 nucleotides of the CACNA1Bsv1 or CACNA1Bsv2 sequence that correspond to a junction polynucleotide region created by the alternative splicing of exon 20 to exon 23 of the primary transcript the CACNA1B gene or by the alternative splicing of exon 21 to exon 23 of the primary transcript the CACNA1B gene, respectively. In each case the CACNA1Bsv1 or CACNA1Bsv2 polynucleotides are selected to comprise a first continuous region of at least 5 to 20 nucleotides that is complementary and hybridizable to the 3′ end of exon 20 or to the 3′ end of exon 21, respectively, and a second continuous region of at least 5 to 20 nucleotides that is complementary and hybridizable to the 5′ of exon 23. As will be apparent to a person of skill in the art, a large number of different polynucleotide sequences from the region of the exon 20 to exon 23 splice junction or from the region of the exon 21 to exon 23 splice junction may be selected which will, under appropriate hybridization conditions, have the capacity to detectably hybridize to CACNA1Bsv1 or CACNA1Bsv2 polynucleotides, respectively, and yet will hybridize to a much less extent to CACNA1B polynucleotides wherein exon 20 or exon 21 is not spliced to exon 23.

Preferably, non-complementary nucleic acid that is present has a particular purpose such as being a reporter sequence or being a capture sequence. However, additional nucleic acid need not have a particular purpose as long as the additional nucleic acid does not prevent the CACNA1Bsv1 or CACNA1Bsv2 nucleic acids from distinguishing between target polynucleotides, e.g., CACNA1Bsv1 or CACNA1Bsv2 polynucleotides and non-target poylnucleotides, including, but not limited to CACNA1B polynucleotides not comprising the exon 20 to exon 23 splice junction found in CACNA1Bsv1 nucleic acids or the exon 21 to exon 23 splice junction found in CACNA1Bsv2 nucleic acids.

Hybridization occurs through complementary nucleotide base pairing. Hybridization conditions determine whether two molecules, or regions, have sufficiently strong interactions with each other to form a stable hybrid.

The degree of interaction between two molecules that hybridize together is reflected by the Tₘ of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and the salt concentration (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×saline sodium citrate (SSC), 5×Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×106 cpm of ³²P-labeled probe. Washing of filters is done at 37° C. for 1 hour in a solution containing 2×SSC, 0.1% SDS. This is followed by a wash in 0.1×SSC, 0.1% sodium dodecyl sulfate (SDS) at 50° C. for 45 minutes before autoradiography. Other procedures using conditions of high stringency would include, for example, either a hybridization step carried out in 5×SSC, 5×Denhardt's solution, 50% formamide at 42° C. for 12 to 48 hours or a washing step carried out in 0.2×saline sodium phosphate-EDTA, 0.2% SDS at 65° C. for 30 to 60 minutes.

Recombinant Expression

CACNA1Bsv1 or CACNA1Bsv2 polynucleotides, such as those comprising SEQ ID NO 1 or SEQ ID NO 3, can be used to make CACNA1Bsv1 or CACNA1Bsv2 polypeptides. In particular, CACNA1Bsv1 or CACNA1Bsv2 polypeptides can be expressed from recombinant nucleic acids in a suitable host or in a test tube using a translation system. Recombinantly expressed CACNA1Bsv1 or CACNA1Bsv2 polypeptides can be used, for example, in assays to screen for compounds that bind to or interact with CACNA1Bsv1 or CACNA1Bsv2 polypeptides, respectively. Alternatively, CACNA1Bsv1 or CACNA1Bsv2 polypeptides can also be used to screen for compounds that bind to or interact with CACNA1Bsv1 or CACNA1Bsv2, respectively, but do not bind to or interact with other isoforms of CACNA1B.

In some embodiments, expression is achieved in a host cell using an expression vector. An expression vector contains recombinant nucleic acid encoding for a polypeptide along with regulatory elements for proper transcription and translation and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXT1 (Stratagene), pSG5 (Stratagene), pCMVLac1 (Stratagene), pCI-neo (Promega), EBO-pSV2-neo (ATCC 37593), pBPV-1 (8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460), and. Bacterial expression vectors well known in the art include pETI11a (Novagen), pBluescript SK (Stratagene, La Jolla), pQE-9 (Qiagen Inc., Valencia), lambda gt11(Invitrogen), pcDNAII (Invitrogen), and pKK223-3 (Pharmacia). Fungal cell expression vectors well known in the art include pPICZ (Invitrogen) and pYES2 (Invitrogen), Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) and MRC-5 (ATCC CCL 171) and HEK 293 cells.

To enhance expression in a particular host it may be useful to modify the sequence provided in SEQ ID NO 1 or SEQ ID NO 3 to take into account codon usage of the host. Codon usage of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acid encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection.

CACNA1Bsv1 AND CACNA1Bsv2 Polypeptides

CACNA1Bsv1 or CACNA1Bsv2 polypeptides contain an amino acid sequence comprising or consisting of SEQ ID NO 2 or SEQ ID NO 4, respectively. CACNA1Bsv1 or CACNA1Bsv2 polypeptides have a variety of uses, such as, for example, providing a marker for the presence of CACNA1Bsv1 or CACNA1Bsv2 polypeptides, respectively; being used as an immunogen to produce antibodies binding to CACNA1Bsv1 or CACNA1Bsv2, respectively; being used as a target polypeptide to identify compounds binding selectively to CACNA1Bsv1 or CACNA1Bsv2 polypeptides, respectively; or being used in an assay to identify compounds that bind to or interact with other isoforms of CACNA1B, but do not bind to or interact with CACNA1Bsv1 or CACNA1Bsv2, respectively.

In chimeric polypeptides containing one or more regions from CACNA1Bsv1 or CACNA1Bsv2 and one or more regions not from CACNA1Bsv1 or CACNA1Bsv2, respectively, the region(s) not from CACNA1Bsv1 or CACNA1Bsv2 can be used, for example, to achieve a particular purpose or to produce a polypeptide that can substitute for CACNA1Bsv1 or CACNA1Bsv2 or fragments thereof. Particular purposes that can be achieved using chimeric CACNA1Bsv1 or CACNA1Bsv2 polypeptides include providing a marker for CACNA1Bsv1 or CACNA1Bsv2 activities, respectively, enhancing an immune response, and modulating neurotransmitter activity.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional CACNA1Bsv1 and CACNA1Bsv2

Functional CACNA1Bsv1 or CACNA1Bsv2 are protein isoforms of CACNA1B. The identification of the amino acid and nucleic acid sequences of CACNA1Bsv1 and CACNA1Bsv2 provide tools for obtaining functional proteins related to CACNA1Bsv1 or CACNA1Bsv2, respectively, from other sources, for producing CACNA1Bsv1 or CACNA1Bsv2 chimeric proteins, and for producing other functional derivatives of SEQ ID NO 2 or SEQ ID NO 4.

CACNA1Bsv1 or CACNA1Bsv2 polypeptides can be readily identified and obtained based on their sequence similarity to CACNA1Bsv1 (SEQ ID NO 2) or CACNA1Bsv2 (SEQ ID NO 4), respectively. In particular, CACNA1Bsv1 polypeptides lack the amino acids encoded by exons 21 and exon 22 of the CACNA1B gene, and CACNA1Bsv2 polypeptides lack the amino acids encoded by exon 22 of the CACNA1B gene. Both the amino acid and nucleic acid sequences of CACNA1Bsv1 or CACNA1Bsv2 can be used to help identify and obtain CACNA1Bsv1 or CACNA1Bsv2 polypeptides, respectively. For example, SEQ ID NO 1 can be used to produce degenerative nucleic acid probes or primers for identifying and cloning nucleic acid polynucleotides encoding for a CACNA1Bsv1 polypeptide. In addition, polynucleotides comprising or consisting of SEQ ID NO 1 or fragments thereof, can also be used under conditions of moderate stringency to identify and clone nucleic acid encoding CACNA1Bsv1 polypeptides from a variety of different organisms. The same methods can also be performed with polynucleotides comprising or consisting of SEQ ID NO 3 or fragments thereof to identify and clone nucleic acids encoding CACNA1Bsv2 polypeptides.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2nd Edition, Cold Spring Harbor Laboratory Press, 1989.

Starting with CACNA1Bsv1 or CACNA1Bsv2 obtained from a particular source, derivatives can be produced. Such derivatives include polypeptides with amino acid substitutions, additions and deletions. Changes to CACNA1Bsv1 or CACNA1Bsv2 to produce a derivative having essentially the same properties should be made in a manner not altering the tertiary structure of CACNA1Bsv1 or CACNA1Bsv2 polypeptides, respectively.

Differences in naturally occurring amino acids are due to different side chain groups. A side chain group produces different properties of the amino acid such as physical size, charge, and hydrophobicity. Amino acids are divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

CACNA1Bsv1 and CACNA1Bsv2 Antibodies

Antibodies recognizing CACNA1Bsv1 or CACNA1Bsv2 can be produced using a polypeptide comprising SEQ ID NO 2 or SEQ ID NO 4 or fragments thereof as immunogens, respectively. Preferably, a CACNA1Bsv1 polypeptide used as an immunogen consists of a polypeptide derived from SEQ ID NO 2 or fragments thereof of having at least 10 contiguous amino acids in length encoded by a polynucleotide region representing the junction resulting from the splicing of exon 20 to exon 23 of the CACNA1B gene. When a CACNA1Bsv2 polypeptide is used as an immunogen, preferably it consists of a polypeptide derived from SEQ ID NO 4 or fragments thereof of having at least 10 contiguous amino acids in length encoded by a polynucleotide region representing the junction resulting from the splicing of exon 21 to exon 23 of the CACNA1B gene.

In some embodiments where, for example, CACNA1Bsv1 polypeptides are used to develop antibodies that bind specifically to CACNA1Bsv1 and not to CACNA1B, the CACNA1Bsv1 polypeptides comprise at least 10 contiguous amino acids of CACNA1Bsv1 encoded by a junction polynucleotide region created by the alternative splicing of exon 20 to exon 23 of the primary transcript of the CACNA1B gene (see FIG. 2). For example, the amino acid sequence: amino terminus-LCSFLRLVRM-carboxy terminus [SEQ ID NO 7], represents one embodiment of such an inventive CACNA1Bsv1 polypeptide wherein the first 5 amino acid region is encoded by nucleotide sequence at the 3′ end of exon 20 of the CACNA1B gene and a second 5 amino acid region is encoded by nucleotides at the 5′ end of exon 23 (see FIG. 2). Preferably, at least 10 amino acids of the CACNA1Bsv1 polypeptide comprises a first continuous region of 2 to 8 amino acids that are encoded by nucleotides at the 3′ end of exon 20 and a second continuous region of 2 to 8 amino acids that are encoded by nucleotides at the 5′ end exon 23.

In other embodiments where, for example, CACNA1Bsv2 polypeptides are used to develop antibodies that bind specifically to CACNA1Bsv2 and not to CACNA1B, the CACNA1Bsv2 polypeptides comprise at least 10 contiguous amino acids of CACNA1Bsv2 encoded by a junction polynucleotide region created by the alternative splicing of exon 21 to exon 23 of the primary transcript the CACNA1B gene (see FIG. 3). For example, the amino acid sequence: amino terminus-YDPAACAWFA-carboxy terminus [SEQ ID NO 8], represents one embodiment of such an inventive CACNA1Bsv2 polypeptide wherein the first 6 amino acid region is encoded by a nucleotide sequence at the 3′ end of exon 21 of the CACNA1B gene and a second 4 amino acid region is encoded by nucleotides at the 5′ end of exon 23 (see FIG. 3). Preferably, at least 10 amino acids of the CACNA1Bsv2 polypeptide comprises a first continuous region of 6 to 8 amino acids that are encoded by nucleotides at the 3′ end of exon 21 and a second continuous region of 2 to 4 amino acids that are encoded by nucleotides at the 5′ end exon 23.

In other embodiments, CACNA1Bsv1-specific antibodies are made using an CACNA1Bsv1 polypeptides that comprise at least 20, 30, 40 or 50 amino acids of the CACNA1Bsv1 sequences that correspond to a junction polynucleotide region created by the alternative splicing of exon 20 to exon 23 in CACNA1Bsv1 in the primary transcript the CACNA1B gene. In each case the CACNA1Bsv1 polypeptides are selected to comprise a first continuous region of at least 5 to 15 amino acids that are encoded by nucleotides at the 3′ end of exon 20 and a second continuous region of 5 to 15 amino acids that are encoded by nucleotides at the 5′ end of exon 23 of CACNA1B and a second continuous region of 5 to 15 amino acids that are encoded by nucleotides at the 5′ end of exon 23 of CACNA1B.

Antibodies to CACNA1Bsv1 or CACNA1Bsv2 have different uses such as being used to identify the presence of CACNA1Bsv1 or CACNA1Bsv2 polypeptides, respectively, and to isolate CACNA1Bsv1 or CACNA1Bsv2 polypeptides, respectively. Identifying the presence of CACNA1Bsv1 can be used, for example, to identify cells producing CACNA1Bsv1. Such identification provides an additional source of CACNA1Bsv1 and can be used to distinguish cells known to produce CACNA1Bsv1 from cells that do not produce CACNA1Bsv1. For example, antibodies to CACNA1Bsv1 can distinguish human cells expressing CACNA1Bsv1 proteins or polypeptides from human cells not expressing CACNA1Bsv1 or non-human cells (including bacteria) that do not express CACNA1Bsv1. Such CACNA1Bsv1 antibodies can also be used to determine the effectiveness of CACNA1By1 ligands, using techniques well known in the art, to detect and quantify changes in the protein levels of CACNA1Bsv1 in cellular extracts, and in situ immunostaining of cells and tissues. In addition, the same above-described utilities also exist for CACNA1Bsv2-specific antibodies.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

CACNA1Bsv1 or CACNA1Bsv2 Binding Assays

A number of compounds known to act as agonists or antagonists of CACNA1B calcium channel activity have been disclosed (see for example, U.S. Pat. No.: 5,646,149, 5,646,145, 6,294,533, 6,423,689). Methods for screening these compounds for their effects on calcium channel activity have also been disclosed (see for example, U.S. Pat. No. 6,096,514). In addition, some organic calcium channel blocking compounds have been described as being useful to treat stroke, cerebral ischemia, head trauma, or epilepsy involving calcium channel activity (see U.S. Pat. No.: 6,294,533, 6,423,689). Therefore, a person skilled in the art can use methods known in the art, such as described in the references cited above, to screen for compounds that bind to, and in some cases functional alter, CACNA1B isoform proteins, or polypeptide fragments thereof.

CACNA1Bsv1, CACNA1Bsv2 or a fragments thereof, can be used in binding studies to identify compounds binding to or interacting with CACNA1Bsv1 or CACNA1Bsv2 or fragments thereof. In one embodiment, CACNA1Bsv1 or fragments thereof can be used in binding studies with CACNA1B protein or a fragment thereof, to identify compounds that: bind to or interact with CACNA1Bsv1 and other CACNA1B isoforms; and bind to or interact with one or more other CACNA1B isoforms and not with CACNA1Bsv1. A similar series of compound screens can, of course, also be performed using CACNA1Bsv2 rather than, or in addition to CACNA1Bsv2. Such binding studies can be performed using different formats including competitive and non-competitive formats. Further competition studies can be carried out using additional compounds determined to bind to CACNA1Bsv1, CACNA1Bsv2 or other CACNA1B isoforms.

The particular CACNA1Bsv1 or CACNA1Bsv2 amino acid sequences involved in ligand binding can be identified by using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

Preferably, binding studies are performed using CACNA1Bsv1 expressed from a recombinant nucleic acid. More preferably, recombinantly expressed CACNA1Bsv1 comprises or consists of the SEQ ID NO 2 amino acid sequence. In addition, binding studies performed using CACNA1Bsv2 are done using protein obtained by expression of the protein from a recombinant nucleic acid. In this case it is preferably that recombinantly expressed CACNA1Bsv2 comprises or consists of the SEQ ID NO 4 amino acid sequence.

Binding assays can be performed using individual compounds or preparations containing different numbers of compounds. A preparation containing different numbers of compounds having the ability to bind to CACNA1Bsv1 or CACNA1Bsv2 can be divided into smaller groups of compounds that can be tested to identify the individual compound(s) binding to either CACNA1Bsv1 or CACNA1Bsv2, respectively.

Binding assays can be performed using recombinantly produced CACNA1Bsv1 or CACNA1Bsv2 polypeptides present in different environments. Such environments include, for example, cell extracts and purified cell extracts containing the CACNA1Bsv1 or CACNA1Bsv2 recombinant nucleic acids; and also include, for example, the use of a purified CACNA1Bsv1 or CACNA1Bsv2 polypeptides produced by recombinant means which are introduced into different environments.

In one embodiment of the invention, a binding method is provided for screening for compounds able to bind selectively to CACNA1Bsv1. The method comprises the steps: providing CACNA1Bsv1 comprising SEQ ID NO 2; providing a CACNA1B isoform polypeptide that is not CACNA1Bsv1, contacting the CACNA1Bsv1 and the CACNA1B isoform polypeptide that is not CACNA1Bsv1 with a test preparation comprising one or more compounds; and then determining the binding of the test preparation to the CACNA1Bsv1 and the CACNA1B isoform polypeptide that is not CACNA1Bsv1, wherein a test preparation that binds to CACNA1Bsv1 but does not bind to CACNA1B isoform polypeptide that is not CACNA1Bsv1 contains one or more compounds that selectively bind to CACNA1Bsv1.

In another embodiment of the invention, a binding method is provided for screening for compounds able to bind selectively to CACNA1Bsv2. The method comprises the steps: providing CACNA1Bsv2 comprising SEQ ID NO 4; providing a CACNA1B isoform polypeptide that is not CACNA1Bsv2, contacting the CACNA1Bsv2 and the CACNA1B isoform polypeptide that is not CACNA1Bsv2 with a test preparation comprising one or more compounds; and then determining the binding of the test preparation to the CACNA1Bsv2 and the CACNA1B isoform polypeptide that is not CACNA1Bsv2, wherein a test preparation that binds to CACNA1Bsv2 but does not bind to CACNA1B isoform polypeptide that is not CACNA1Bsv2 contains one or more compounds that selectively bind to CACNA1Bsv2.

In another embodiment of the invention, a binding method is provided for screening for compounds able to bind selectively to a CACNA1B isoform polypeptide that is not CACNA1Bsv1. The method comprises the steps of: providing CACNA1Bsv1 comprising SEQ ID NO 2; providing a CACNA1B isoform polypeptide that is not CACNA1Bsv1, contacting CACNA1Bsv1 and CACNA1B isoform polypeptide that is not CACNA1Bsv1 with a test preparation comprising one or more compounds; and then determining the binding of the test preparation to CACNA1Bsv1 and CACNA1B isoform polypeptide that is not CACNA1Bsv1, wherein a test preparation that binds CACNA1B isoform polypeptide that is not CACNA1Bsv1 but does not bind to CACNA1Bsv1 contains a compound that selectively binds the CACNA1B isoform polypeptide that is not CACNA1Bsv1. Alternatively, the above method can be used to identify compounds that bind selectively to a CACNA1B isoform polypeptide that is not CACNA1Bsv2 by performing the method with CACNA1Bsv2 protein comprising SEQ ID NO 4.

The above-described selective binding assays can also be performed with polypeptide fragments of CACNA1Bsv1 or CACNA1Bsv2, wherein the polypeptide fragments comprise at least 10 consecutive amino acids that are encoded by nucleotide sequences that bridge the junction created by the splicing of the 3′ end of exon 20 to the 5′ end of exon 23 in the case of CACNA1Bsv1, or the splicing of the 3′ end of exon 21 to the 5′ end of exon 23 in the case of CACNA1Bsv2. Similarly, the selective binding assays may also be performed using a polypeptide fragments of a CACNA1B isoform polypeptide that is not CACNA1Bsv1 or CACNA1Bsv2 wherein the polypeptide fragments comprise at least 10 consecutive amino acids that are encoded by: a) a nucleotide sequence that is contained within exon 22 of CACNA1B; b) a nucleotide sequence that bridges the junction created by the splicing of the 3′ end of exon 20 to the 5′ end of exon 21 of CACNA1B; or c) a nucleotide sequence that bridges the junction created by the splicing of the 3′ end of exon 22 to the 5′ end of exon 23 of CACNA1B.

Calcium Channel (CACNA1B) Functional Assays

The activity of a calcium channel may be assessed in vitro by methods known to those of skill in the art, including the electrophysiological methods (e.g., Williams et al., 1992 Science 257, 389-395; see also, for example, U.S. Pat. Nos. 6,353,091; 6,156,726; and 6,096,514). Typically, calcium channel α-subunit polypeptides include regions with which one or more modulators of calcium channel activity, such as 1,4-dihydropyridine (1,4-DHP) or omega-conotoxin (ω-CgTx), interact directly or indirectly. Types of α subunits, e.g., CACNA1A verses CACNA1B, may be distinguished by any method known to those skilled in the art, including on the basis of binding specificity. For example, CACNA1B polypeptides participates in the formation of channels that have previously been referred to as N-type channels. The activity of channels that contain CACNA1B is insensitive to 1,4-DHP and is irreversibly blocked by ω-CgTx. Omega-conotoxins are a family of peptide toxins of 24 to 30 amino acids that are known to block N-type calcium channels or N/P/Q-type calcium channels (Adams et al., 1999 Drug Dev. Res. 46, 219-234;). N-type calcium channel specific ω-CgTxs include, for example, GVIA (isolated from Conus geographus venom), CVIA and CVID (isolated from Conus catus venom), TVIA (isolated from Conus tulipa venom), and MVIIA (isolated from Conus magus venom) (Lewis et al., 2000 J. Biol. Chem. 275, 35335-35344).

Isoforms of CACNA1B may also be characterized on the basis of the effects of modulators on the subunit or differences in electrical currents and current kinetics produced by calcium channels containing CACNA1B subunits. The identification of CACNA1Bsv1 and CACNA1Bsv2 as splice variants isoforms of CACNA1B provides a means for screening for compounds that bind to CACNA1Bsv1 or CACNA1Bsv2 calcium channels using the methods of toxin sensitivity and/or electrical current patterns. Assays involving a functional CACNA1Bsv1 or CACNA1Bsv2 polypeptide can be employed for different purposes, such as, for example, selecting for compounds that effect or alter CACNA1Bsv1 or CACNA1Bsv2 functional activity, respectively, and mapping the activity of different CACNA1Bsv1 or CACNA1Bsv2 polypeptide regions. CACNA1B isoform activity can be measured using different techniques such as: detecting a change in the intracellular conformation of CACNA1Bsv1 or CACNA1Bsv2; detecting a change in the intracellular location of CACNA1Bsv1 or CACNA1Bsv2; detecting the amount of binding of 1,4-DHP or ω-CgTx; or measuring differences in electrical currents and current kinetics produced by calcium channels containing CACNA1Bsv1 or CACNA1Bsv2 subunits.

Techniques for measuring CACNA1B-mediated calcium channel activity are available to the person skilled in the art. In particular, mammalian HEK tissue culture cells have been transiently and stably transfected with DNA comprising one or more human calcium channel subunits (Williams et al., 1992 Science 257: 389395). Such transfected cells express heterologous calcium channels that exhibit pharmacological and electrophysiological properties that can be ascribed to human calcium channels. Such cells, however, represent homogeneous populations and the pharmacological and electrophysiological data obtained therefrom provides measurements of human calcium channel activity. For example, HEK cells can be transiently transfected with DNA comprising the CACNA1Bsv1 or CACNA1Bsv2 splice variants. The resulting cells transiently express the target CACNA1B isoform polypeptide, which form calcium channels that have properties that may be pharmacologically distinct from other voltage-activated N-type calcium channels, e.g., may exhibit altered sensitivity to ω-conotoxin and have electrical currents that are different from other CACNA1B isoform calcium channels. For example, it has been found that alteration of the molar ratios of different calcium channel subunits introduced into the cells to achieve equivalent mRNA levels significantly increased the number of receptors per cell, the current density, and affected the ω-conotoxin sensitivity (Hans et al., 1999, Biophys. J. 76, 1384-1400).

The effects of compounds that bind or interact with calcium channels formed by variants CACNA1Bsv1 or CACNA1Bsv2 can be assessed using cells expressing CACNA1Bsv1 or CACNA1Bsv2 proteins, respectively, that are then contacted with individual compounds or test preparations containing one or more different compounds. A test preparation containing different compounds which is found to affect CACNA1Bsv1 or CACNA1Bsv2 activity in cells that overproduce CACNA1Bsv1 or CACNA1Bsv2, respectively, as compared to control cells containing an expression vector lacking CACNA1Bsv1 or CACNA1Bsv2 coding sequence, can then be divided into smaller groups of compounds to identify the compound(s) that is affecting CACNA1Bsv1 or CACNA1Bsv2 activity.

CACNA1B isoform functional assays can be performed using recombinantly produced CACNA1B isoform polypeptides, such as CACNA1Bsv1 or CACNA1Bsv2, present in different environments. Such environments include, for example, cell extracts and purified cell extracts containing the target CACNA1B isoform polypeptide expressed from a recombinant nucleic acid encoding the target CACNA1B isoform and an appropriate membrane for the polypeptide; and the use of purified target CACNA1B isoform proteins or polypeptides thereof produced by recombinant means that are introduced into a different environment suitable for measuring calcium channel activity.

Modulating CACNA1Bsv1 OR CACNA1Bsv2 Expression

CACNA1Bsv1 or CACNA1Bsv2 proteins or polypeptides expression can be modulated as a means for increasing or decreasing CACNA1Bsv1 or CACNA1Bsv2 activities, respectively. Such modulation includes inhibiting the activity of nucleic acids encoding the CACNA1B isoform target to reduce CACNA1B isoform protein or polypeptide expressions, or supplying CACNA1B nucleic acids to increase the level of expression of the CACNA1B target polypeptide thereby increasing target calcium channel activity.

Inhibition of CACNA1Bsv1 or CACNA1Bsv2 Activities

CACNA1Bsv1 or CACNA1Bsv2 nucleic acid activities can be inhibited using anti-sense nucleic acids recognizing CACNA1Bsv1 or CACNA1Bsv2 nucleic acids, respectively, and affecting the ability of such nucleic acids to be transcribed or translated. Inhibition of CACNA1Bsv1 or CACNA1Bsv2 nucleic acid activities can be used, for example, in target validation studies.

A preferred target for inhibiting CACNA1Bsv1 or CACNA1Bsv2 is mRNA translation. The ability of CACNA1Bsv1 mRNA or CACNA1Bsv2 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing the processing of mRNA, hybrid arrest, and degradation of mRNA by RNAse H activity.

RNAi also can be used to prevent protein expression of a target transcript. This method is based on the interfering properties of double-stranded RNA derived from the coding regions of gene that disrupts the synthesis of protein from transcribed RNA.

Enzymatic nucleic acid can recognize and cleave another nucleic acid molecule. Preferred enzymatic nucleic acids are ribozymes.

General structures for anti-sense nucleic acids, RNAi and ribozymes, and methods of delivering such molecules, are well known in the art. Modified and unmodified nucleic acids can be used as anti-sense molecules, RNAi and ribozymes. Different types of modifications can effect certain anti-sense activities such as the ability to be cleaved by RNAse H, and can effect nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999 Cell 99:123-32; Fire, et al., 1998 Nature 391:806-11), plants (Hamilton and Baulcombe, 1999 Science 286:950-52), Drosophila (Hammond, et al., 2001 Science 293:1146-50; Misquitta and Patterson, 1999 Proc. Nat. Acad. Sci. 96:1451-56; Kennerdell and Carthew, 1998 Cell 95:1017-26), and mammalian cells (Bernstein, et al., 2001 Nature 409:363-6; Elbashir, et al., 2001 Nature 411:494-8).

Increasing CACNA1Bsv1 or CACNA1Bsv2 Expressions

Nucleic acids coding for CACNA1Bsv1 or CACNA1Bsv2 can be used, for example, to cause an increase in Ca²⁺ channel activity or to create a test system (e.g., a transgenic animal) for screening for compounds affecting the expression of CACNA1Bsv1 or CACNA1Bsv2, respectively. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra. Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of CACNA1Bsv1 and CACNA1Bsv2 Using Tiling Microarrays

To identify variants of the “normal” splicing of the exon regions in CACNA1B, an exon junction microarray, comprising probes complementary to each predicted splice junction resulting from splicing of CACNA1B exons in heteronuclear RNA (hnRNA), was hybridized to a mixture of cRNAs prepared from samples obtained from 39 different human tissues and one tissue sample obtained from monkey. Exon junction microarrays are described in PCT patent applications WO 02/18646 and WO 02/16650. Materials and methods for preparing hybridization samples from purified RNA, hybridizing the microarrays, detecting hybridization signals, and data analysis are described in van't Veer, et al. (2002 Nature 415:530-6); Hughes, et al. (2001 Nature Biotechnol. 19:342-7) and Hughes, et al. (2000 Cell 102:109-26). Inspection of the exon junction microarray hybridization data (not shown) suggested that the structure of at least one of the exon 21 or exon 22 junctions of CACNAB mRNA was altered in a large number of tissues examined, suggesting the presence of at least one CACNA1B splice variant mRNA population within the normal CACNA1B mRNA population. RT-PCR was then performed using oligonucleotide primers complementary to exons 19 and 25 to confirm the exon junction array results and to allow the sequence structure of the putative splice variant(s) to be determined.

### Example 2

### Confirmation of CACNA1Bsv1 and CACNA1Bsv2 Using RT-PCR

The structure of CACNA1B mRNA in the region coding for exon 19 to exon 25 was determined for a panel of human tissues using a reverse transcription and polymerase chain reaction (RT-PCR) based assay. PolyA purified mRNA isolated from 39 different human tissues was obtained from BD Biosciences Clontech (Palo Alto, Calif.), Biochain Institute, Inc. (Hayward, Calif.), and Ambion Inc. (Austin, Tex.). In addition, one monkey brain mRNA sample (from Biochain Institute, Inc.) was also obtained and assayed. RT-PCR primers of 28 nucleotides were selected that were complementary to sequences in exons 19 and 25 in CACNA1B (NM_{—000718}). Based upon the nucleotide sequence of CACNA1B mRNA, the CACNA1B exon 19 and exon 25 primer set (hereafter CACNA1B 19-25 primer set) was expected to amplify a 700 base pair amplicon representing “normal” CACNA1B mRNA region comprising exon 19 to exon 25 (see FIGS. 2 and 3). The CACNA1B exon 19 primer has the sequence: 5′ GTTTGGGAATATTGCCCTGGATGATGAC 3′ [SEQ ID NO 9]; and the CACNA1B exon 25 primer has the sequence: 5′CTTCCCCACTGTCATCTCA TCAGGCTTA 3′ [SEQ ID NO 10].

Twenty-five ng of polyA mRNA from each tissue was subjected to a one-step reverse transcription-PCR amplification protocol using the Qiagen, Inc. (Valencia, Calif.), One-Step RT-PCR kit, using the following conditions:

Cycling conditions were as follows:

50° C. for 30 minutes;

95° C. for 15 minutes;

35 cycles of:

95° C. for 1 minutes;

60° C. for 1 minutes;

72° C. for 1 minutes; then

72° C. for 15 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were sequenced from each end (using the same primers used for RT-PCR) by Qiagen Genomics, Inc. (Bothell, Wash.).

At least five different RT-PCR amplicons were obtained from human mRNA samples using the CACNA1B₁₉₋₂₅ primer set. Every human tissue evaluated exhibited the expected amplicon size of ˜700 base pairs for normally spliced CACNA1B mRNA. In addition, the monkey brain mRNA sample also exhibited the expected ˜700 base pair amplicon. However, in addition to the expected CACNA1B amplicon of ˜700 base pairs, the human testes tissues also exhibited multiple amplicons. The testes RT-PCR reaction yielded five visible DNA fragments. Counting from largest (top, band 1) to smallest (lowest band). Bands 2-5 were purified and sequenced. Band 2 was the expected long-form (700 bp); band 3 was determined to be 603 base pairs (CACNA1Bsv2); the sequence of band 4 could not be determined and band 5 was determined to be 475 base pairs (CACNA1Bsv1).

Sequence analysis of the 603 base pair and 475 base pair amplicons of CACNA1B revealed that these amplicon forms are due to alternative splicing of exon 21 of CACNA1B hnRNA to exon 23 in the case of the 603 base pair amplicon (CACNA1Bsv2), and, splicing of exon 20 of CACNA1B hnRNA to exon 23 in the case of the 475 base pair amplicon (CACNA1Bsv1). That is, the shorter forms of CACNA1B amplicons were due to the complete absence of exons 21 and 22 of the coding sequence of CACNA1B in the case of the 475 base pair amplicon and the complete absence of exon 22 of the coding sequence of CACNA1B in the case of the 603 base pair amplicon. Thus, the RT-PCR results confirmed the junction probe microarray data reported in Example 1, which suggested that CACNA1B mRNA was composed of a mixed population of molecules in some human tissue samples wherein in the CACNA1B mRNA was alternately spliced. The tissues in which CACNA1Bsv1 and CACNA1Bsv2 mRNAs were detected are listed in Table 1.

TABLE 1

| Sample | CACNA1Bsv1 | CACNA1Bsv2 |
|---|---|---|
| Salivary Gland, | | |
| Ileocecum | | |
| Liver, left lobe | | |
| Epididymus | | |
| Peripheral leukocytes | | |
| Fetal skeletal muscle | | |
| Melanoma (G361) | | |
| Brain-cerebellum | X | X |
| Brain-pons | X | X |
| Brain, monkey | | X |
| Tonsil | | |
| Ileum | | |
| Fetal liver | | |
| Testes | X | X |
| Lymph node | | |
| Skeletal muscle | | |
| Burkitt's lymphoma (Raji) | | |
| Brain, thalamus | | X |
| Brain, parietal lobe | | X |
| Fetal spinal cord | | |
| Tongue | | |
| Jejunum | | |
| Liver | | |
| Prostate | | |
| Thymus | | |
| Retina | | |
| Colorectal adenocarcinoma (SW480) | | |
| Brain, corpus callosum | | X |
| Brain, occipital lobe | | X |
| Spinal Cord | X | |
| Fetal heart | | |
| Duodenum | | |
| Fetal kidney | | |
| Thyroid | | |
| Spleen | | |
| Adipose tissue | | |
| Chronic Myclogenous leukemia (K562) | | |
| Brain, caudate nucleus | | X |
| Brain, medulla oblongata | | X |
| Brain, paracentral gyrus | | X |

### Example 3

### Cloning of CACNA1Bsv1 and CACNA1Bsv2

Microarray and RT-PCR data indicated that in addition to normal CACNA1B mRNA sequence, NM_{—000718}, encoding CACNA1B protein, NP_{—000709}), at least two different splice variant forms of CACNA1B mRNA also exists in some human tissues.

A full length CACNA1B clone having a nucleotide sequence comprising the “475 base pair short form” splice variant (hereafter referred to as CACNA1Bsv1) or comprising the “603 base pair short form” splice variant (hereafter referred to as CACNA1Bsv2), as identified in Example 2, are isolated using a 5′ “forward” CACNA1B primer and a 3′ “reverse” CACNA1B primer, to amplify and clone the entire mRNA coding sequences encoding either CACNA1Bsv1 or CACNA1Bsv2 isoform proteins. The 5′ “forward” CACNA1B primer for both CACNA1Bsv1 and CACNA1Bsv2 is designed to have a nucleotide of 5′ ATGGTCCGCTTCGGGGACGAGCTGGG 3′ (SEQ ID NO 11). The 3′ “reverse” CACNA1Bsv1 primer is designed to have the nucleotide sequence of 5′ GCACCAGTGGTCTTGGTCAGGGTGGT 3′ (SEQ ID NO 12). The 3′ “reverse” CACNA1Bsv2 primer is designed to have the nucleotide sequence of 5′ TGCGAACCAGGCGCACGCAGCCGGGT 3′ (SEQ ID NO 13).

RT-PCR

A CACNA1Bsv1 or CACNA1Bsv2 cDNA sequence is cloned using a combination of reverse transcription (RT) and polymerase chain reaction (PCR). More specifically, about 25 ng of human testes polyA mRNA (Ambion, Austin, Tex.) is reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction is added to 40 μl of water, 5 μl of 10× buffer, 1 μl of dNTPs and 1 μl of enzyme from the Clontech (PaloAlto, Calif.) Advantage 2 PCR kit. PCR is done in a Gene Amp PCR System 9700 (Applied Biosystems, Foster City, Calif.) using the CACNA1B “forward” and “reverse” primers. After an initial 94° C. denaturation of 1 minute, 30 cycles of amplification are performed using a 15 second denaturation at 95° C. followed by a 7 minute synthesis at 68° C. The 30 cycles of PCR are followed by a 10 minute extension at 68° C. The 50 μl reaction is then chilled to 4° C. 10 μL1 of the resulting reaction product is run on a 1% agarose (Invitrogen, Ultra pure) gel stained with 0.3 μg/ml ethidium bromide (Fisher Biotech, Fair Lawn, N.J.). The gel is visualized and photographed on a UV light box to determined if the PCR has yielded products of the expected size, in the case of the predicted CACNA1Bsv1 mRNA, a product of about 6.31 kilobases (Kb) and CACNA1Bsv2 mRNA 6.44 kB. In practice, both the CACNA1Bsv1 and the CACNA1Bsv2 RT-PCR products are purified in the same gel fragment, in addition to the RT-PCR product corresponding to CACNA1B. A fragment estimated to be about 6.5 kilobases (Kb) is extracted from the gel and purified with a QIAquick Gel Extraction kit (Qiagen, Valencia, Calif.).

Cloning of RT-PCR Products

About 4 μl of the 6 μl of purified mixed CACNA1B RT-PCR products from testes are used in a cloning reaction using the reagents and instructions provided with the TOPO XL PCR Cloning Kit (Invitrogen, Carlsbad, Calif.). About 2 μl of the cloning reaction is used following the manufacturer's instructions to transform TOP10 chemically competent E. coli provided with the cloning kit. After the 1 hour recovery of the cells in SOC medium (provided with the TOPO XL PCR Cloning Kit), 200 μl of the mixture is plated on LB medium plates (Sambrook, et al., in Molecular Cloning, A Laboratory Manual, 2^{nd} Edition, Cold Spring Harbor Laboratory Press, 1989) containing 50 μg/ml Kanamycin (Sigma, St. Louis, Mo.). Plates are incubated overnight at 37° C. Twenty colonies are picked from the plates into 2 ml of LB medium containing 50 μg kanamycin/ml. These liquid cultures are incubated overnight on a roller at 37° C. Plasmid DNA is extracted from these cultures using the Qiagen (Valencia, Calif.) Qiaquik Spin Miniprep kit.

Plasmid DNA purified from the twenty putative CACNA1Bsv1 clones or the twenty CACNA1Bsv2 clones identified above as having the expected insert structures is subjected to PCR using the CACNA1B1₁₉₋₂₅ primer set. Clones having the CACNA1Bsv1 or CACNA1Bsv2 structures are identified based upon amplification of an amplicon band of 126 base pairs or 257 base pairs, whereas a normal CACNA1B clone will give rise to an amplicon band of 351 base pairs. DNA sequence analysis of insert DNA from each of the CACNA1Bsv1 and CACNA1Bsv2 clones produce a polynucleotide sequence of CACNA1Bsv1 (SEQ ID NO 1) and CACNA1Bsv2 (SEQ ID NO 3).

SEQ ID NO 1 has an open reading frame that encodes CACNA1Bsv1 protein (SEQ ID NO 2). CACNA1Bsv1 is identical to CACNA1B (NP_{—}000709), but lacks a 75 amino acids region encoded by exon 21 and exon 22 of CACNA1B (NM_{—}000718).

SEQ ID NO 3 has an open reading frame that encodes CACNA1Bsv2 protein (SEQ ID NO 4). CACNA1Bsv2 is identical to CACNA1B (NP_{—}000850) up to and through most of the coding sequence of exon 21. However, the alternative splicing of the coding sequence of exon 21 to exon 23 not only drops the 97 base pairs of exon 22, but also results in the creation of a protein translation reading frame that is out of alignment with the normal CACNA1B exon 23 protein reading frame. This shift in reading frame at exon 23 in the CACNA1Bsv2 mRNA, results in the production of a truncated CACNA1Bsv2 isoform protein as compared to CACNA1B (NP_{—}000709).

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

What is claimed:

1. A purified human nucleic acid comprising SEQ ID NO 1, or the complement thereof.

2. The purified nucleic acid of claim 1, wherein said nucleic acid comprises a polynucleotide encoding for the amino acid sequence of SEQ ID NO 2.

3. The purified nucleic acid of claim 1, wherein said nucleotide sequence encodes for a polypeptide consisting of the amino acid sequence of SEQ ID NO 2.

4. A purified polypeptide comprising SEQ ID NO 2.

5. The polypeptide of claim 4, wherein said polypeptide consists of the amino acid sequence of SEQ ID NO 2.

6. An expression vector comprising a nucleotide sequence encoding for SEQ ID NO 2, wherein said nucleotide sequence is transcriptionally coupled to an exogenous promoter.

7. The expression vector of claim 6, wherein said nucleotide sequence encodes for a polypeptide consisting of the amino acid sequence of SEQ ID NO 2.

8. The expression vector of claim 6, wherein said nucleotide sequence comprises SEQ ID NO 1.

9. The expression vector of claim 6, wherein said nucleotide sequence consists of the sequence of SEQ ID NO 1.

10. A recombinant cell comprising the expression vector of claim 6, wherein said cell comprises an RNA polymerase recognized by said promoter.

11. A recombinant cell made by a process comprising the step of introducing the expression vector of claim 6 into said cell.

12. A method of preparing a CACNA1Bsv1 polypeptide comprising the step of growing the recombinant cell of claim 10 under conditions wherein said polypeptide is expressed from said expression vector.

13. A method of screening for compounds able to bind selectively to CACNA1Bsv1 comprising the steps of:
(a) providing a CACNA1Bsv1 polypeptide comprising SEQ ID NO 2;
(b) providing a CACNA1B polypeptide that is not CACNA1Bsv1,
(c) contacting said CACNA1Bsv1 polypeptide and said CACNA1B polypeptide that is not CACNA1Bsv1 with a test preparation comprising one or more compounds; and
(d) determining the binding of said test preparation to said CACNA1Bsv1 polypeptide and said CACNA1B polypeptide that is not CACNA1Bsv1, wherein a test preparation which binds said CACNA1Bsv1 polypeptide but does not bind said CACNA1B polypeptide that is not CACNA1Bsv1 contains a compound that selectively binds said CACNA1Bsv1 polypeptide.

14. The method of claim 13, wherein said CACNA1Bsv1 polypeptide is obtained by expression of said polypeptide from an expression vector comprising a polynucleotide encoding the amino acid sequence of SEQ ID NO 2.

15. The method of claim 14, wherein said polypeptide consists of the amino acid sequence of SEQ ID NO 2.

16. A method of screening for a compound able to bind to or interact with a CACNA1Bsv1 protein or a fragment thereof comprising the steps of:
(a) expressing a CACNA1Bsv1 polypeptide comprising the amino acid sequence of SEQ ID NO 2 or fragment thereof from a recombinant nucleic acid;
(b) providing to said polypeptide a labeled CACNA1B ligand that binds to said polypeptide and a test preparation comprising one or more compounds; and
(c) measuring the effect of said test preparation on binding of said labeled CACNA1B ligand to said polypeptide, wherein a test preparation that alters the binding of said labeled CACNA1B ligand to said polypeptide contains a compound that binds to or interacts with said polypeptide.

17. The method of claim 16, wherein said steps (b) and (c) are performed in vitro.

18. The method of claim 16, wherein said steps (a), (b) and (c) are preformed using a whole cell.

19. The method of claim 16, wherein said polypeptide is expressed from an expression vector.

20. The method of claim 16, wherein said CACNA1B ligand is a calcium channel-binder.

21. The method of claim 20, wherein said polypeptide consists of an amino acid sequence provided for in SEQ ID NO 1 or a fragment of SEQ ID NO 1.

22. The method of claim 20, wherein said test preparation contains one compound.


SEQUENCE LISTING (13 sequences)

SEQ ID NO: 1 - DNA, 6792 bp, Homo sapiens
atggtccgcttcggggacgagctgggcggccgctatggaggccccggcggcggagagcgggcccggggcggcggggccggcggggcggggggcccgggtcccggggggctgcagcccggccagcgggtcctctacaagcaatcgatcgcgcagcgcgcgcggaccatggcgctgtacaaccccatcccggtcaagcagaactgcttcaccgtcaaccgctcgctcttcgtcttcagcgaggacaacgtcgtccgcaaatacgcgaagcgcatcaccgagtggcctccattcgagtatatgatcctggccaccatcatcgccaactgcatcgtgctggccctggagcagcacctccctgatggggacaaaacgcccatgtccgagcggctggacgacacggagccctatttcatcgggatcttttgcttcgaggcagggatcaaaatcatcgctctgggctttgtcttccacaagggctcttacctgcggaacggctggaacgtcatggacttcgtggtcgtcctcacagggatccttgccacggctggaactgacttcgacctgcgaacactgagggctgtgcgtgtgctgaggcccctgaagctggtgtctgggattccaagtttgcaggtggtgctcaagtccatcatgaaggccatggttccactcctgcagattgggctgcttctcttctttgccatcctcatgtttgccatcattggcctggagttctacatgggcaagttccacaaggcctgtttccccaacagcacagatgcggagcccgtgggtgacttcccctgtggcaaggaggccccagcccggctgtgcgagggcgacactgagtgccgggagtactggccaggacccaactttggcatcaccaactttgacaatatcctgtttgccatcttgacggtgttccagtgcatcaccatggagggctggactgacatcctctataatacaaacgatgcggccggcaacacctggaactggctctacttcatccctctcatcatcatcggctccttcttcatgctcaacctggtgctgggcgtgctctcgggggagtttgccaaggagcgagagagggtggagaaccgccgcgccttcctgaagctgcgccggcagcagcagatcgagcgagagctcaacgggtacctggagtggatcttcaaggcggaggaagtcatgctggccgaggaggacaggaatgcagaggagaagtcccctttggacgtgctgaagagagcggccaccaagaagagcagaaatgacctgatccacgcagaggagggagaggaccggtttgcagatctctgtgctgttggatcccccttcgcccgcgccagcctcaagagcgggaagacagagagctcgtcatacttccggaggaaggagaagatgttccggttttttatccggcgcatggtgaaggctcagagcttctactgggtggtgctgtgcgtggtggccctgaacacactgtgtgtggccatggtgcattacaaccagccgcggcggcttaccacgaccctgtattttgcagagtttgttttcctgggtctcttcctcacagagatgtccctgaagatgtatggcctggggcccagaagctacttccggtcctccttcaactgcttcgactttggggtcatcgtggggagcgtctttgaagtggtctgggcggccatcaagccgggaagctcctttgggatcagtgtgctgcgggccctccgcctgctgaggatcttcaaagtcacgaagtactggagctccctgcggaacctggtggtgtccctgctgaactccatgaagtccatcatcagcctgctcttcttgctcttcctgttcattgtggtcttcgccctgctggggatgcagctgtttgggggacagttcaacttccaggatgagactcccacaaccaacttcgacaccttccctgccgccatcctcactgtcttccagatcctgacgggagaggactggaatgcagtgatgtatcacgggatcgaatcgcaaggcggcgtcagcaaaggcatgttctcgtccttttacttcattgtcctgacactgttcggaaactacactctgctgaatgtctttctggccatcgctgtggacaacctggccaacgcccaagagctgaccaaggatgaagaggagatggaagaagcagccaatcagaagcttgctctgcaaaaggccaaagaagtggctgaagtcagccccatgtctgccgcgaacatctccatcgccgccaggcagcagaactcggccaaggcgcgctcggtgtgggagcagcgggccagccagctacggctgcagaacctgcgggccagctgcgaggcgctgtacagcgagatggaccccgaggagcggctgcgcttcgccactacgcgccacctgcggcccgacatgaagacgcacctggaccggccgctggtggtggagctgggccgcgacggcgcgcgggggcccgtgggaggcaaagcccgacctgaggctgcggaggcccccgagggcgtcgaccctccgcgcaggcaccaccggcaccgcgacaaggacaagacccccgcggcgggggaccaggaccgagcagaggccccgaaggcggagagcggggagcccggtgcccgggaggagcggccgcggccgcaccgcagccacagcaaggaggccgcggggcccccggaggcgcggagcgagcgcggccgaggcccaggccccgagggcggccggcggcaccaccggcgcggctccccggaggaggcggccgagcgggagccccgacgccaccgcgcgcaccggcaccaggatccgagcaaggagtgcgccggcgccaagggcgagcggcgcgcgcggcaccgcggcggcccccgagcggggccccgggaggcggagagcggggaggagccggcgcggcggcaccgggcccggcacaaggcgcagcctgctcacgaggctgtggagaaggagaccacggagaaggaggccacggagaaggaggctgagatagtggaagccgacaaggaaaaggagctccggaaccaccagccccgggagccacactgtgacctggagaccagtgggactgtgactgtgggtcccatgcacacactgcccagcacctgtctccagaaggtggaggaacagccagaggatgcagacaatcagcggaacgtcactcgcatgggcagtcagcccccagacccgaacactattgtacatatcccagtgatgctgacgggccctcttggggaagccacggtcgttcccagtggtaacgtggacctggaaagccaagcagaggggaagaaggaggtggaagcggatgacgtgatgaggagcggcccccggcctatcgtcccatacagctccatgttctgtttaagccccaccaacctgctccgccgcttctgccactacatcgtgaccatgaggtacttcgaggtggtcattctcgtggtcatcgccttgagcagcatcgccctggctgctgaggacccagtgcgcacagactcgcccaggaacaacgctctgaaatacctggattacattttcactggtgtctttacctttgagatggtgataaagatgatcgacttgggactgctgcttcaccctggagcctatttccgggacttgtggaacattctggacttcattgtggtcagtggcgccctggtggcgtttgctttctcaggatccaaagggaaagacatcaataccatcaagtctctgagagtccttcgtgtcctgcggcccctcaagaccatcaaacggctgcccaagctcaaggctgtgtttgactgtgtggtgaactccctgaagaatgtcctcaacatcttgattgtctacatgctcttcatgttcatatttgccgtcattgcggtgcagctcttcaaagggaagtttttctactgcacagatgaatccaaggagctggagagggactgcaggggtcagtatttggattatgagaaggaggaagtggaagctcagcccaggcagtggaagaaatacgactttcactacgacaatgtgctctgggctctgctgacgctgttcacagtgtccacgggagaaggctggcccatggtgctgaaacactccgtggatgccacctatgaggagcagggtccaagccctgggtaccgcatggagctgtccatcttctacgtggtctactttgtggtctttcccttcttcttcgtcaacatctttgtggctttgatcatcatcaccttccaggagcagggggacaaggtgatgtctgaatgcagcctggagaagaacgagagggcttgcattgacttcgccatcagcgccaaacccctgacacggtacatgccccaaaaccggcagtcgttccagtataagacgtggacatttgtggtctccccgccctttgaatacttcatcatggccatgatagccctcaacactgtggtgctgatgatgaagttctatgatgcaccctatgagtacgagctgatgctgaaatgcctgaacatcgtgttcacatccatgttctccatggaatgcgtgctgaagatcatcgcctttggggtgctgaactatttcagagatgcctggaatgtctttgactttgtcactgtgttgggaagtattactgatattttagtaacagagattgcggaaacgaacaatttcatcaacctcagcttcctccgcctctttcgagctgcgcggctgatcaagctgctccgccagggctacaccatccgcatcctgctgtggacctttgtccagtccttcaaggccctgccctacgtgtgtctgctcattgccatgctgttcttcatctacgccatcatcggcatgcaggtgtttgggaatattgccctggatgatgacaccagcatcaaccgccacaacaacttccggacgtttttgcaagccctgatgctgctgttcaggagcgccacgggggaggcctggcacgagatcatgctgtcctgcctgagcaaccaggcctgtgatgagcaggccaatgccaccgagtgtggaagtgactttgcctacttctacttcgtctccttcatcttcctgtgctcctttctgcgcctggttcgcatgaacatgcccatctccaacgaggacatgactgttcacttcacgtccacgctgatggccctcatccggacggcactggagatcaagctggccccagctgggacaaagcagcatcagtgtgacgcggagttgaggaaggagatttccgttgtgtgggccaatctgccccagaagactttggacttgctggtaccaccccataagcctgatgagatgacagtggggaaggtttatgcagctctgatgatatttgacttctacaagcagaacaaaaccaccagagaccagatgcagcaggctcctggaggcctctcccagatgggtcctgtgtccctgttccaccctctgaaggccaccctggagcagacacagccggctgtgctccgaggagcccgggttttccttcgacagaagagttccacctccctcagcaatggcggggccatacaaaaccaagagagtggcatcaaagagtctgtctcctggggcactcaaaggacccaggatgcaccccatgaggccaggccacccctggagcgtggccactccacagagatccctgtggggcggtcaggagcactggctgtggacgttcagatgcagagcataacccggaggggccctgatggggagccccagcctgggctggagagccagggtcgagcggcctccatgccccgccttgcggccgagactcagcccgtcacagatgccagccccatgaagcgctccatctccacgctggcccagcggccccgtgggactcatctttgcagcaccaccccggaccgcccaccccctagccaggcgtcgtcgcaccaccaccaccaccgctgccaccgccgcagggacaggaagcagaggtccctggagaaggggcccagcctgtctgccgatatggatggcgcaccaagcagtgctgtggggccggggctgcccccgggagaggggcctacaggctgccggcgggaacgagagcgccggcaggagcggggccggtcccaggagcggaggcagccctcatcctcctcctcggagaagcagcgcttctactcctgcgaccgctttgggggccgtgagcccccgaagcccaagccctccctcagcagccacccaacgtcgccaacagctggccaggagccgggaccccacccacagggcagtggttccgtgaatgggagccccttgctgtcaacatctggtgctagcacccccggccgcggtgggcggaggcagctcccccagacgcccctgactccccgccccagcatcacctacaagacggccaactcctcacccatccacttcgccggggctcagaccagcctccctgccttctccccaggccggctcagccgtgggctttccgaacacaacgccctgctgcagagagaccccctcagccagcccctggcccctggctctcgaattggctctgacccttacctggggcagcgtctggacagtgaggcctctgtccacgccctgcctgaggacacgctcactttcgaggaggctgtggccaccaactcgggccgctcctccaggacttcctacgtgtcctccctgacctcccagtctcaccctctccgccgcgtgcccaacggttaccactgcaccctgggactcagctcgggtggccgagcacggcacagctaccaccaccctgaccaagaccactggtgc

SEQ ID NO: 2 - protein, 2264 aa, Homo sapiens
MVRFGDELGGRYGGPGGGERARGGGAGGAGGPGPGGLQPGQRVLYKQSIAQRARTMALYNPIPVKQNCFTVNRSLFVFSEDNVVRKYAKRITEWPPFEYMILATIIANCIVLALEQHLPDGDKTPMSERLDDTEPYFIGIFCFEAGIKIIALGFVFHKGSYLRNGWNVMDFVVVLTGILATAGTDFDLRTLRAVRVLRPLKLVSGIPSLQVVLKSIMKAMVPLLQIGLLLFFAILMFAIIGLEFYMGKFHKACFPNSTDAEPVGDFPCGKEAPARLCEGDTECREYWPGPNFGITNFDNILFAILTVFQCITMEGWTDILYNTNDAAGNTWNWLYFIPLIIIGSFFMLNLVLGVLSGEFAKERERVENRRAFLKLRRQQQIERELNGYLEWIFKAEEVMLAEEDRNAEEKSPLDVLKRAATKKSRNDLIHAEEGEDRFADLCAVGSPFARASLKSGKTESSSYFRRKEKMFRFFIRRMVKAQSFYWVVLCVVALNTLCVAMVHYNQPRRLTTTLYFAEFVFLGLFLTEMSLKMYGLGPRSYFRSSFNCFDFGVIVGSVFEVVWAAIKPGSSFGISVLRALRLLRIFKVTKYWSSLRNLVVSLLNSMKSIISLLFLLFLFIVVFALLGMQLFGGQFNFQDETPTTNFDTFPAAILTVFQILTGEDWNAVMYHGIESQGGVSKGMFSSFYFIVLTLFGNYTLLNVFLAIAVDNLANAQELTKDEEEMEEAANQKLALQKAKEVAEVSPMSAANISIAARQQNSAKARSVWEQRASQLRLQNLRASCEALYSEMDPEERLRFATTRHLRPDMKTHLDRPLVVELGRDGARGPVGGKARPEAAEAPEGVDPPRRHHRHRDKDKTPAAGDQDRAEAPKAESGEPGAREERPRPHRSHSKEAAGPPEARSERGRGPGPEGGRRHHRRGSPEEAAEREPRRHRAHRHQDPSKECAGAKGERRARHRGGPRAGPREAESGEEPARRHRARHKAQPAHEAVEKETTEKEATEKEAEIVEADKEKELRNHQPREPHCDLETSGTVTVGPMHTLPSTCLQKVEEQPEDADNQRNVTRMGSQPPDPNTIVHIPVMLTGPLGEATVVPSGNVDLESQAEGKKEVEADDVMRSGPRPIVPYSSMFCLSPTNLLRRFCHYIVTMRYFEVVILVVIALSSIALAAEDPVRTDSPRNNALKYLDYIFTGVFTFEMVIKMIDLGLLLHPGAYFRDLWNILDFIVVSGALVAFAFSGSKGKDINTIKSLRVLRVLRPLKTIKRLPKLKAVFDCVVNSLKNVLNILIVYMLFMFIFAVIAVQLFKGKFFYCTDESKELERDCRGQYLDYEKEEVEAQPRQWKKYDFHYDNVLWALLTLFTVSTGEGWPMVLKHSVDATYEEQGPSPGYRMELSIFYVVYFVVFPFFFVNIFVALIIITFQEQGDKVMSECSLEKNERACIDFAISAKPLTRYMPQNRQSFQYKTWTFVVSPPFEYFIMAMIALNTVVLMMKFYDAPYEYELMLKCLNIVFTSMFSMECVLKIIAFGVLNYFRDAWNVFDFVTVLGSITDILVTEIAETNNFINLSFLRLFRAARLIKLLRQGYTIRILLWTFVQSFKALPYVCLLIAMLFFIYAIIGMQVFGNIALDDDTSINRHNNFRTFLQALMLLFRSATGEAWHEIMLSCLSNQACDEQANATECGSDFAYFYFVSFIFLCSFLRLVRMNMPISNEDMTVHFTSTLMALIRTALEIKLAPAGTKQHQCDAELRKEISVVWANLPQKTLDLLVPPHKPDEMTVGKVYAALMIFDFYKQNKTTRDQMQQAPGGLSQMGPVSLFHPLKATLEQTQPAVLRGARVFLRQKSSTSLSNGGAIQNQESGIKESVSWGTQRTQDAPHEARPPLERGHSTEIPVGRSGALAVDVQMQSITRRGPDGEPQPGLESQGRAASMPRLAAETQPVTDASPMKRSISTLAQRPRGTHLCSTTPDRPPPSQASSHHHHHRCHRRRDRKQRSLEKGPSLSADMDGAPSSAVGPGLPPGEGPTGCRRERERRQERGRSQERRQPSSSSSEKQRFYSCDRFGGREPPKPKPSLSSHPTSPTAGQEPGPHPQGSGSVNGSPLLSTSGASTPGRGGRRQLPQTPLTPRPSITYKTANSSPIHFAGAQTSLPAFSPGRLSRGLSEHNALLQRDPLSQPLAPGSRIGSDPYLGQRLDSEASVHALPEDTLTFEEAVATNSGRSSRTSYVSSLTSQSHPLRRVPNGYHCTLGLSSGGRARHSYHHPDQDHWC

SEQ ID NO: 3 - DNA, 5235 bp, Homo sapiens
atggtccgcttcggggacgagctgggcggccgctatggaggccccggcggcggagagcgggcccggggcggcggggccggcggggcggggggcccgggtcccggggggctgcagcccggccagcgggtcctctacaagcaatcgatcgcgcagcgcgcgcggaccatggcgctgtacaaccccatcccggtcaagcagaactgcttcaccgtcaaccgctcgctcttcgtcttcagcgaggacaacgtcgtccgcaaatacgcgaagcgcatcaccgagtggcctccattcgagtatatgatcctggccaccatcatcgccaactgcatcgtgctggccctggagcagcacctccctgatggggacaaaacgcccatgtccgagcggctggacgacacggagccctatttcatcgggatcttttgcttcgaggcagggatcaaaatcatcgctctgggctttgtcttccacaagggctcttacctgcggaacggctggaacgtcatggacttcgtggtcgtcctcacagggatccttgccacggctggaactgacttcgacctgcgaacactgagggctgtgcgtgtgctgaggcccctgaagctggtgtctgggattccaagtttgcaggtggtgctcaagtccatcatgaaggccatggttccactcctgcagattgggctgcttctcttctttgccatcctcatgtttgccatcattggcctggagttctacatgggcaagttccacaaggcctgtttccccaacagcacagatgcggagcccgtgggtgacttcccctgtggcaaggaggccccagcccggctgtgcgagggcgacactgagtgccgggagtactggccaggacccaactttggcatcaccaactttgacaatatcctgtttgccatcttgacggtgttccagtgcatcaccatggagggctggactgacatcctctataatacaaacgatgcggccggcaacacctggaactggctctacttcatccctctcatcatcatcggctccttcttcatgctcaacctggtgctgggcgtgctctcgggggagtttgccaaggagcgagagagggtggagaaccgccgcgccttcctgaagctgcgccggcagcagcagatcgagcgagagctcaacgggtacctggagtggatcttcaaggcggaggaagtcatgctggccgaggaggacaggaatgcagaggagaagtcccctttggacgtgctgaagagagcggccaccaagaagagcagaaatgacctgatccacgcagaggagggagaggaccggtttgcagatctctgtgctgttggatcccccttcgcccgcgccagcctcaagagcgggaagacagagagctcgtcatacttccggaggaaggagaagatgttccggttttttatccggcgcatggtgaaggctcagagcttctactgggtggtgctgtgcgtggtggccctgaacacactgtgtgtggccatggtgcattacaaccagccgcggcggcttaccacgaccctgtattttgcagagtttgttttcctgggtctcttcctcacagagatgtccctgaagatgtatggcctggggcccagaagctacttccggtcctccttcaactgcttcgactttggggtcatcgtggggagcgtctttgaagtggtctgggcggccatcaagccgggaagctcctttgggatcagtgtgctgcgggccctccgcctgctgaggatcttcaaagtcacgaagtactggagctccctgcggaacctggtggtgtccctgctgaactccatgaagtccatcatcagcctgctcttcttgctcttcctgttcattgtggtcttcgccctgctggggatgcagctgtttgggggacagttcaacttccaggatgagactcccacaaccaacttcgacaccttccctgccgccatcctcactgtcttccagatcctgacgggagaggactggaatgcagtgatgtatcacgggatcgaatcgcaaggcggcgtcagcaaaggcatgttctcgtccttttacttcattgtcctgacactgttcggaaactacactctgctgaatgtctttctggccatcgctgtggacaacctggccaacgcccaagagctgaccaaggatgaagaggagatggaagaagcagccaatcagaagcttgctctgcaaaaggccaaagaagtggctgaagtcagccccatgtctgccgcgaacatctccatcgccgccaggcagcagaactcggccaaggcgcgctcggtgtgggagcagcgggccagccagctacggctgcagaacctgcgggccagctgcgaggcgctgtacagcgagatggaccccgaggagcggctgcgcttcgccactacgcgccacctgcggcccgacatgaagacgcacctggaccggccgctggtggtggagctgggccgcgacggcgcgcgggggcccgtgggaggcaaagcccgacctgaggctgcggaggcccccgagggcgtcgaccctccgcgcaggcaccaccggcaccgcgacaaggacaagacccccgcggcgggggaccaggaccgagcagaggccccgaaggcggagagcggggagcccggtgcccgggaggagcggccgcggccgcaccgcagccacagcaaggaggccgcggggcccccggaggcgcggagcgagcgcggccgaggcccaggccccgagggcggccggcggcaccaccggcgcggctccccggaggaggcggccgagcgggagccccgacgccaccgcgcgcaccggcaccaggatccgagcaaggagtgcgccggcgccaagggcgagcggcgcgcgcggcaccgcggcggcccccgagcggggccccgggaggcggagagcggggaggagccggcgcggcggcaccgggcccggcacaaggcgcagcctgctcacgaggctgtggagaaggagaccacggagaaggaggccacggagaaggaggctgagatagtggaagccgacaaggaaaaggagctccggaaccaccagccccgggagccacactgtgacctggagaccagtgggactgtgactgtgggtcccatgcacacactgcccagcacctgtctccagaaggtggaggaacagccagaggatgcagacaatcagcggaacgtcactcgcatgggcagtcagcccccagacccgaacactattgtacatatcccagtgatgctgacgggccctcttggggaagccacggtcgttcccagtggtaacgtggacctggaaagccaagcagaggggaagaaggaggtggaagcggatgacgtgatgaggagcggcccccggcctatcgtcccatacagctccatgttctgtttaagccccaccaacctgctccgccgcttctgccactacatcgtgaccatgaggtacttcgaggtggtcattctcgtggtcatcgccttgagcagcatcgccctggctgctgaggacccagtgcgcacagactcgcccaggaacaacgctctgaaatacctggattacattttcactggtgtctttacctttgagatggtgataaagatgatcgacttgggactgctgcttcaccctggagcctatttccgggacttgtggaacattctggacttcattgtggtcagtggcgccctggtggcgtttgctttctcaggatccaaagggaaagacatcaataccatcaagtctctgagagtccttcgtgtcctgcggcccctcaagaccatcaaacggctgcccaagctcaaggctgtgtttgactgtgtggtgaactccctgaagaatgtcctcaacatcttgattgtctacatgctcttcatgttcatatttgccgtcattgcggtgcagctcttcaaagggaagtttttctactgcacagatgaatccaaggagctggagagggactgcaggggtcagtatttggattatgagaaggaggaagtggaagctcagcccaggcagtggaagaaatacgactttcactacgacaatgtgctctgggctctgctgacgctgttcacagtgtccacgggagaaggctggcccatggtgctgaaacactccgtggatgccacctatgaggagcagggtccaagccctgggtaccgcatggagctgtccatcttctacgtggtctactttgtggtctttcccttcttcttcgtcaacatctttgtggctttgatcatcatcaccttccaggagcagggggacaaggtgatgtctgaatgcagcctggagaagaacgagagggcttgcattgacttcgccatcagcgccaaacccctgacacggtacatgccccaaaaccggcagtcgttccagtataagacgtggacatttgtggtctccccgccctttgaatacttcatcatggccatgatagccctcaacactgtggtgctgatgatgaagttctatgatgcaccctatgagtacgagctgatgctgaaatgcctgaacatcgtgttcacatccatgttctccatggaatgcgtgctgaagatcatcgcctttggggtgctgaactatttcagagatgcctggaatgtctttgactttgtcactgtgttgggaagtattactgatattttagtaacagagattgcggaaacgaacaatttcatcaacctcagcttcctccgcctctttcgagctgcgcggctgatcaagctgctccgccagggctacaccatccgcatcctgctgtggacctttgtccagtccttcaaggccctgccctacgtgtgtctgctcattgccatgctgttcttcatctacgccatcatcggcatgcaggtgtttgggaatattgccctggatgatgacaccagcatcaaccgccacaacaacttccggacgtttttgcaagccctgatgctgctgttcaggagcgccacgggggaggcctggcacgagatcatgctgtcctgcctgagcaaccaggcctgtgatgagcaggccaatgccaccgagtgtggaagtgactttgcctacttctacttcgtctccttcatcttcctgtgctcctttctgatgttgaacctctttgtggctgtgatcatggacaattttgagtacctcacgcgggactcttccatcctaggtcctcaccacttggatgagttcatccgggtctgggctgaatacgacccggctgcgtgcgcctggttcgca

SEQ ID NO: 4 - protein, 1745 aa, Homo sapiens
MVRFGDELGGRYGGPGGGERARGGGAGGAGGPGPGGLQPGQRVLYKQSIAQRARTMALYNPIPVKQNCFTVNRSLFVFSEDNVVRKYAKRITEWPPFEYMILATIIANCIVLALEQHLPDGDKTPMSERLDDTEPYFIGIFCFEAGIKIIALGFVFHKGSYLRNGWNVMDFVVVLTGILATAGTDFDLRTLRAVRVLRPLKLVSGIPSLQVVLKSIMKAMVPLLQIGLLLFFAILMFAIIGLEFYMGKFHKACFPNSTDAEPVGDFPCGKEAPARLCEGDTECREYWPGPNFGITNFDNILFAILTVFQCITMEGWTDILYNTNDAAGNTWNWLYFIPLIIIGSFFMLNLVLGVLSGEFAKERERVENRRAFLKLRRQQQIERELNGYLEWIFKAEEVMLAEEDRNAEEKSPLDVLKRAATKKSRNDLIHAEEGEDRFADLCAVGSPFARASLKSGKTESSSYFRRKEKMFRFFIRRMVKAQSFYWVVLCVVALNTLCVAMVHYNQPRRLTTTLYFAEFVFLGLFLTEMSLKMYGLGPRSYFRSSFNCFDFGVIVGSVFEVVWAAIKPGSSFGISVLRALRLLRIFKVTKYWSSLRNLVVSLLNSMKSIISLLFLLFLFIVVFALLGMQLFGGQFNFQDETPTTNFDTFPAAILTVFQILTGEDWNAVMYHGIESQGGVSKGMFSSFYFIVLTLFGNYTLLNVFLAIAVDNLANAQELTKDEEEMEEAANQKLALQKAKEVAEVSPMSAANISIAARQQNSAKARSVWEQRASQLRLQNLRASCEALYSEMDPEERLRFATTRHLRPDMKTHLDRPLVVELGRDGARGPVGGKARPEAAEAPEGVDPPRRHHRHRDKDKTPAAGDQDRAEAPKAESGEPGAREERPRPHRSHSKEAAGPPEARSERGRGPGPEGGRRHHRRGSPEEAAEREPRRHRAHRHQDPSKECAGAKGERRARHRGGPRAGPREAESGEEPARRHRARHKAQPAHEAVEKETTEKEATEKEAEIVEADKEKELRNHQPREPHCDLETSGTVTVGPMHTLPSTCLQKVEEQPEDADNQRNVTRMGSQPPDPNTIVHIPVMLTGPLGEATVVPSGNVDLESQAEGKKEVEADDVMRSGPRPIVPYSSMFCLSPTNLLRRFCHYIVTMRYFEVVILVVIALSSIALAAEDPVRTDSPRNNALKYLDYIFTGVFTFEMVIKMIDLGLLLHPGAYFRDLWNILDFIVVSGALVAFAFSGSKGKDINTIKSLRVLRVLRPLKTIKRLPKLKAVFDCVVNSLKNVLNILIVYMLFMFIFAVIAVQLFKGKFFYCTDESKELERDCRGQYLDYEKEEVEAQPRQWKKYDFHYDNVLWALLTLFTVSTGEGWPMVLKHSVDATYEEQGPSPGYRMELSIFYVVYFVVFPFFFVNIFVALIIITFQEQGDKVMSECSLEKNERACIDFAISAKPLTRYMPQNRQSFQYKTWTFVVSPPFEYFIMAMIALNTVVLMMKFYDAPYEYELMLKCLNIVFTSMFSMECVLKIIAFGVLNYFRDAWNVFDFVTVLGSITDILVTEIAETNNFINLSFLRLFRAARLIKLLRQGYTIRILLWTFVQSFKALPYVCLLIAMLFFIYAIIGMQVFGNIALDDDTSINRHNNFRTFLQALMLLFRSATGEAWHEIMLSCLSNQACDEQANATECGSDFAYFYFVSFIFLCSFLMLNLFVAVIMDNFEYLTRDSSILGPHHLDEFIRVWAEYDPAACAWFA

SEQ ID NO: 5 - DNA, 39 bp, Homo sapiens
tcttcctgtgctcctttctcgcctggttcgcatgaacat

SEQ ID NO: 6 - DNA, 40 bp, Homo sapiens
gaatacgacccggctgcgtgcgcctggttcgcatgaacat

SEQ ID NO: 7 - protein, 10 aa, Homo sapiens
LCSFLRLVRM

SEQ ID NO: 8 - protein, 10 aa, Homo sapiens
YDPAACAWFA

SEQ ID NO: 9 - DNA, 28 bp, Homo sapiens
gtttgggaatattgccctggatgatgac

SEQ ID NO: 10 - DNA, 28 bp, Homo sapiens
cttccccactgtcatctcatcaggctta

SEQ ID NO: 11 - DNA, 26 bp, Homo sapiens
atggtccgcttcggggacgagctggg

SEQ ID NO: 12 - DNA, 26 bp, Homo sapiens
gcaccagtggtcttggtcagggtggt

SEQ ID NO: 13 - DNA, 26 bp, Homo sapiens
tgcgaaccaggcgcacgcagccgggt